SEQUENCE LISTING (14 sequences)

SEQ ID NO: 1 - protein, 124 aa, Artificial Sequence
  Synthetic polypeptide
QVQLQQSGPGLVKPSQTLSLTCAISGDSVSSNSAAWGWIRQSPGRGLEWLGRIYYRSKWYNSYAVSVKSRITINPDTSKNQFSLQLNSVTPEDTAVYYCARYDWVPKIGVFDSWGQGTLVTVSS

SEQ ID NO: 2 - protein, 108 aa, Artificial Sequence
  sYNTHETIC POLYPEPTIDE
DIVLTQSPATLSLSPGERATLSCRASQFISSSYLSWYQQKPGQAPRLLIYGSSSRATGVPARFSGSGSGTDFTLTISSLEPEDFAVYYCQQLYSSPMTFGQGTKVEIK

SEQ ID NO: 3 - protein, 12 aa, Artificial Sequence
  Synthetic polypeptide
GDSVSSNSAAWG

SEQ ID NO: 4 - protein, 18 aa, Artificial Sequence
  sYNTHETIC POLYPEPTIDE
RIYYRSKWYNSYAVSVKS

SEQ ID NO: 5 - protein, 12 aa, Artificial Sequence
  Synthetic polypeptide
YDWVPKIGVFDS

SEQ ID NO: 6 - protein, 12 aa, Artificial Sequence
  sYNTHETIC POLYPEPTIDE
RASQFISSSYLS

SEQ ID NO: 7 - protein, 7 aa, Artificial Sequence
  Synthetic polypeptide
GSSSRAT

SEQ ID NO: 8 - protein, 9 aa, Artificial Sequence
  sYNTHETIC POLYPEPTIDE
QQLYSSPMT

SEQ ID NO: 9 - protein, 454 aa, Artificial Sequence
  Synthetic polypeptide
QVQLQQSGPGLVKPSQTLSLTCAISGDSVSSNSAAWGWIRQSPGRGLEWLGRIYYRSKWYNSYAVSVKSRITINPDTSKNQFSLQLNSVTPEDTAVYYCARYDWVPKIGVFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 215 aa, Artificial Sequence
  sYNTHETIC POLYPEPTIDE
DIVLTQSPATLSLSPGERATLSCRASQFISSSYLSWYQQKPGQAPRLLIYGSSSRATGVPARFSGSGSGTDFTLTISSLEPEDFAVYYCQQLYSSPMTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 372 bp, Artificial Sequence
  Synthetic polynucleotide
caggtgcagctgcagcagagcggcccaggcctggtcaagccctctcagaccctgtcactgacctgcgccatttcaggcgacagcgtgagcagcaacagcgccgcctggggctggatcaggcagagccccggtaggggcctggaatggctgggcaggatctactacaggtccaagtggtacaacagctacgccgtgagcgtgaagagcaggatcaccatcaaccctgacaccagcaagaaccagttctcactgcagctcaacagcgtgacccccgaggacaccgccgtgtactactgcgccagatacgactgggtgcccaagatcggcgtgttcgacagctggggccagggcaccctggtgaccgtgtcaagc

SEQ ID NO: 12 - DNA, 324 bp, Artificial Sequence
  sYNTHETIC POLYNUCLEOTIDE
gatatcgtgctgacacagagccccgccaccctgagcctgagcccaggcgagagggccaccctgtcctgcagggccagccagtttatcagcagcagctacctgtcctggtatcagcagaagcccggccaggcccctagactgctgatctacggcagctcctctcgggccaccggcgtgcccgccaggttcagcggcagcggctccggcaccgacttcaccctgacaatcagcagcctggagcccgaggacttcgccgtgtactactgccagcagctgtacagctcacccatgaccttcggccagggcaccaaggtggagatcaag

SEQ ID NO: 13 - DNA, 1419 bp, Artificial Sequence
  Synthetic polynucleotide
atggcctgggtgtggaccctgcccttcctgatggccgctgcccagtcagtgcaggcccaggtgcagctgcagcagagcggcccaggcctggtcaagccctctcagaccctgtcactgacctgcgccatttcaggcgacagcgtgagcagcaacagcgccgcctggggctggatcaggcagagccccggtaggggcctggaatggctgggcaggatctactacaggtccaagtggtacaacagctacgccgtgagcgtgaagagcaggatcaccatcaaccctgacaccagcaagaaccagttctcactgcagctcaacagcgtgacccccgaggacaccgccgtgtactactgcgccagatacgactgggtgcccaagatcggcgtgttcgacagctggggccagggcaccctggtgaccgtgtcaagcgccagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 14 - DNA, 705 bp, Artificial Sequence
  sYNTHETIC POLYNUCLEOTIDE
atgagcgtgctgacccaggtgctggctctgctgctgctgtggctgaccggcaccagatgcgatatcgtgctgacacagagccccgccaccctgagcctgagcccaggcgagagggccaccctgtcctgcagggccagccagtttatcagcagcagctacctgtcctggtatcagcagaagcccggccaggcccctagactgctgatctacggcagctcctctcgggccaccggcgtgcccgccaggttcagcggcagcggctccggcaccgacttcaccctgacaatcagcagcctggagcccgaggacttcgccgtgtactactgccagcagctgtacagctcacccatgaccttcggccagggcaccaaggtggagatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

(19) United States
(12) Patent Application Publication
(10) Pub. No.: US 2023/0279132 A1
(43) Pub. Date: Sep. 7, 2023

(54) TREATMENT OF B CELL MALIGNANCIES

(71) Applicant: NOVARTIS AG, Basel (CH)

(72) Inventors: Nadia HASSOUNAH, Livingston, NJ (US); Anwesha CHAUDHURY, Boston, MA (US); Somesh CHOUDHURY, Hillsborough, NJ (US); Janghee WOO, Short Hills, NJ (US); Nicole Anju Punjabi KUNDAMAL, Jersey City, NJ (US)

(21) Appl. No.: 18/019,469

(22) PCT Filed: Aug. 4, 2021
(86) PCT No.: PCT/US2021/044113

§ 371 Date: Feb. 2, 2023

Related U.S. Application Data
(60) Provisional application No. 63/060,786, filed on Aug. 4, 2020.
(60) Provisional application No. 63/114,363, filed on Nov. 16, 2020.
(60) Provisional application No. 63/147,507, filed on Feb. 9, 2021.

(51) Int. Cl. C07K 16/28; A61K 31/454; A61P 35/02

(52) U.S. Cl. CPC C07K 16/2878; A61K 31/454; A61P 35/02; A61K 2039/545

(57) ABSTRACT

The present disclosure relates to anti-BAFFR antibodies and binding fragments thereof, alone or in combination with additional agents, for use in the treatment of B cell malignancies, for example a B-cell non-Hodgkin's lymphoma.

### 1. CROSS-REFERENCE TO RELATED APPLICATIONS

This application claims the priority benefit of U.S. provisional application nos. 63/060,786, filed Aug. 4, 2020, 63/114,363, filed Nov. 16, 2020, and 63/147,507, filed Feb. 9, 2021, the contents of each of which are incorporated herein in their entireties by reference thereto.

### 2. SEQUENCE LISTING

The instant application contains a Sequence Listing which has been submitted electronically in ASCII format and is hereby incorporated by reference in its entirety. Said ASCII copy, created on 26 Jul. 2021, is named PAT059003-WO-PCT_SL.txt and is 13,921 bytes in size.

### 3. FIELD OF INVENTION

The disclosure generally relates to anti-BAFFR antibodies and binding fragments thereof, for use in the treatment of B cell malignancies, such as B-cell non-Hodgkin Lymphomas, as monotherapy or as a “backbone” agent in combination with one or more additional agents.

### 4. BACKGROUND

B cell malignancies include non-Hodgkin lymphomas (NHL), Hodgkin's lymphomas, leukemias, and myeloma.

NHLs account for approximately 4% of all cancers, with an estimated 74,200 new cases diagnosed and about 19,970 deaths in the United States (US) in 2019 (www.cancer.org). NHL are classified by the WHO into immature lymphoid neoplasms, mature B-cell neoplasms, T-cell and NK-cell neoplasms, and post-transplant lymphoproliferative disorders (PTLD). Mature B-cell lymphomas are further classified into indolent lymphomas (e.g. follicular lymphoma, small lymphocytic lymphoma) and aggressive lymphomas (e.g. diffuse large B-cell lymphoma, mantle cell lymphoma).

Diffuse large B-cell lymphoma (DLBCL) is the most frequent aggressive lymphoma subtype representing 30-35% of all NHL (Ghielmini et al 2013). Approximately one-third of DLBCL patients will develop relapsed and/or refractory (r/r) disease, which represents a major cause of morbidity and mortality. Relapsed and/or refractory patients have a poor prognosis. While approximately 60% of patients with r/r DLBCL remain sensitive to conventional second line salvage immuno-chemotherapy, <10% have prolonged disease-free survival (Gisselbrecht et al 2010). In the recently reported SCHOLAR-1 study, which combined data from two clinical trials as well as two academic databases, the median overall survival of 636 patients with refractory DLBCL was only 6.3 months, whereas only 20% of the patients were alive at 2 years (Crump et al 2017). For transplant candidates who fail second line therapy or who relapse post-transplant, prognosis is grave. Novel therapies that showed promising activity include oral targeted therapies, such as the BTK inhibitor ibrutinib (ORR 25% for DLBCL patients) (Wilson et al 2015), monoclonal antibodies (e.g., immune-checkpoint-inhibitors with response rates of ˜40% in DLBCL (Lesokhin et al 2016; Zinzani et al 2017)), bispecific antibodies such as blinatumomab with response rates of 35-50% (Viardot et al 2016), ADCs (ORR 44% for brentuximab vedotin in CD30+ relapsed DLBCL (Jacobsen et al 2015), ORR 70% for polatuzumab vedotin in combination with rituximab and bendamustine (Sehn et al 2017)) and cellular therapies (e.g. CAR-T therapies ORR 53-82% (Neelapu et al 2017; Schuster et al 2017)). CAR-T therapy seems to result in long-term remissions for approximately one third of r/r DLBCL patients. Recently, in 2020, tafasitamab-cxix (MOR208, MONJUVI®) plus lenalidomide (ORR 55%, CR 37%) received initial FDA approval for patients with relapsed or refractory DLBCL who are ineligible for transplants (tafasitamab-cxix package insert).

Mantle cell lymphoma (MCL) is an aggressive mature B-cell non-Hodgkin lymphoma associated with poor long-term survival. Treatment consists primarily of immuno-chemotherapy; therapeutic strategy and choice of regimen is variable and dependent on symptoms and patient fitness (Schieber et al 2018). Despite high initial response rates (ORR>90% for aggressive treatment approaches) MCL remains incurable. 5-year overall survival for low-risk patients is approximately 80% compared with 34% in high-risk patients (Hoster et al 2014). Median survival of relapsed MCL has historically been less than 3 years. Targeted therapies, such as bortezomib (Fisher et al 2006) and temsirolimus (Hess et al 2009) improved response rates. Lenalidomide showed promising activity (28% ORR) in a heavily pre-treated population in the Phase II study EMERGE, leading to its approval in the US (Goy et al 2013). In a Phase III study the Bruton's tyrosine kinase ibrutinib showed a significant improvement in progression-free survival (PFS) compared to temsirolimus, as well as fewer treatment-related adverse effects (Dreyling et al 2016). The phase II data combining ibrutinib with the Bcl-2 inhibitor venetoclax achieved 42% complete response rate (CRR) compared with 9% in historical controls (Tam et al 2018). More recently, data from acalabrutinib single agent phase II study for treatment of relapsed/refractory MCL based on (ACE-LY-004 study) showed a median PFS 20 months, ORR of 81%, 43% CR rate (Wang et al 2019). Despite the promising developments over the last decade, novel therapeutic options are needed for patients with r/r MCL, given the 1-year overall survival rate of approximately 70% in the ibrutinib era (Schieber et al 2018).

In spite of newer therapies being introduced for treatment of B cell malignancies, including relapsed/refractory NHL such as DLBCL and MCL, there is still a high unmet medical need for patients with B cell malignancies, for example patients with relapsed/refractory NHL. These patients have few effective treatment options and significantly reduced overall survival.

### 5. SUMMARY

The disclosure relates to anti-B-cell activating factor receptor antibodies (anti-BAFFR antibodies) and binding fragments thereof for use in the treatment of B cell malignancies (for example a non-Hodgkin's lymphoma such as DLBCL or MCL) as monotherapy or as a “backbone” agent in combination with one or more additional agents, for example, an immunomodulatory imide drug (IMiD) such as lenalidomide. BAFFR is broadly expressed in B cell malignancies such as NHL and CLL, and preclinical and clinical studies with the anti-BAFFR antibody ianalumab (VAY736) reported in the Examples of the subject application support the use of anti-BAFFR antibodies and binding fragments thereof for the treatment of B cell malignancies as monotherapy or in combination with one or more additional agents.

Accordingly, in one aspect, the disclosure provides an anti-BAFFR antibody (e.g., ianalumab) or a binding fragment thereof for use in the treatment of a B cell malignancy in a subject in need thereof, wherein the anti-BAFFR antibody or binding fragment thereof is to be administered at a therapeutically effective dose.

In some embodiments, the anti-BAFFR antibody or binding fragment thereof is to be administered in combination with one or more additional agents. In some embodiments, the one or more additional agents comprise an immunomodulatory imide drug (IMiD) such as lenalidomide.

In another aspect, the disclosure provides new dosing regimens for anti-BAFFR antibodies (e.g., ianalumab) and binding fragments thereof that can be used in methods of treating B cell malignancies.

Exemplary anti-BAFFR antibodies and dosage regimens for use in treating a B cell malignancy are described in Section 7.2 and specific embodiments 1 to 32, infra. Exemplary additional agents are described in Section 7.3 and specific embodiments 33 to 49, infra. Exemplary B cell malignancies and patient populations suitable for treatment using the methods and compositions described herein are described in Section 7.4 and specific embodiments 116 to 166.

In another aspect, the disclosure provides methods of treating subjects having a B cell malignancy with an anti-BAFFR antibody (e.g., ianalumab) or a binding fragment thereof as monotherapy, or in combination with one or more additional agents. Exemplary methods of treatment are described in specific embodiments 50 to 100, infra.

In another aspect, the disclosure provides combinations comprising an anti-BAFFR antibody (e.g., ianalumab) or a binding fragment thereof and one or more additional agents, for example an immunomodulatory imide drug (IMiD). Such combinations can be used, for example, in a method of treating a subject having a B cell malignancy, e.g., as described herein. In some embodiments, a combination comprises ianalumab and lenalidomide. Exemplary combinations are described in specific embodiments 101 to 111, infra.

In another aspect, the disclosure provides for the use of anti-BAFFR antibodies (e.g., ianalumab) and binding fragments thereof and additional agents in the manufacture of medicaments for treating a subject having a B cell malignancy (e.g., a NHL such as DLBCL or MCL). In some embodiments, the subject has a NHL, for example DLBCL or MCL, and (i) has failed at least one prior line (and optionally up to five prior lines) of standard of care therapy, e.g., an anti-CD20 therapy such as rituximab and/or (ii) is intolerant to or ineligible for one or more other approved therapies, e.g., autologous stem cell transplant (ASCT) and/or (iii) is a non-responder to a chimeric antigen receptor (CAR) T cell therapy. The NHL can be relapsed and/or refractory.

In some embodiments, a medicament comprising an anti-BAFFR antibody (e.g., ianalumab) or a binding fragment thereof is for administration in combination with an additional agent, e.g., an additional agent described herein, and/or for administration in a method of treatment described herein. Exemplary uses of anti-BAFFR antibodies and binding fragments thereof and additional agents in the manufacture of medicaments are described in specific embodiments 112 to 115, infra.

In further aspects, the disclosure provides anti-BAFFR antibodies and binding fragments thereof for use in treating a subject with cytokine release syndrome (CRS) or at risk for CRS, methods of reducing the severity of one or more symptoms of CRS in a subject by administering an anti-BAFFR antibody or binding fragment thereof to the subject, and use of anti-BAFFR antibodies and binding fragments thereof in the manufacture of medicaments for treating a subject with CRS or at risk of CRS. Without being bound by theory, it is believed that cytokine release by normal B cells is an important driver in CRS, and it is believed that depleting normal B cells in a subject with an anti-BAFFR antibody or binding fragment thereof can reduce the severity of CRS experienced by a subject and/or reduce the likelihood of developing CRS. Exemplary anti-BAFFR antibodies and binding fragments thereof for use in treating a subject with cytokine release syndrome (CRS) or at risk for CRS, methods of reducing the severity of one or more symptoms of CRS in a subject using an anti-BAFFR antibody or binding fragment thereof, and uses of anti-BAFFR antibodies and binding fragments thereof in the manufacture of medicaments for treating a subject with CRS or at risk of CRS are described in specific embodiments 167 to 170, infra.

### 6. BRIEF DESCRIPTION OF THE FIGURES

FIGS. 1A-1D: Tumor growth in an in vivo model of DLBCL in animals treated with vehicle (FIG. 1A), VAY736 at 5 mg/kg (FIG. 1B), VAY736 50 mg/kg (FIG. 1C) or rituximab (FIG. 1D).

FIGS. 2A-2B: Percent specific lysis of SUDHL4 cells in the presence of VAY736 by NK3.3 cells treated with lenalidomide (L) for 72 hr. NK3.3 cells were treated with lenalidomide or DMSO for 72 hr, then added to Calcein AM-labeled SUDHL4 cells for 2 hr in the presence of a dose curve of VAY736, at an E:T of 10:1 (FIG. 2A) or 20:1 (FIG. 2B). A total of 7.5e3 SUDHL4 cells were plated. The X-axis is the concentration (ng/ml) of VAY736.

FIGS. 3A-3C: Percent specific lysis of SUDHL4 cells in the presence of VAY736 by PBMC treated with lenalidomide (L) for 24 hr. Unstimulated PBMC were treated with lenalidomide or DMSO for 24 hr, then added to Calcein AM-labeled SUDHL4 cells for 4 hr in the presence of a dose curve of VAY736, at an E:T of 2:1 (FIG. 3A), 10:1 (FIG. 3B) or 20:1 (FIG. 3C). A total of 7.5e3 SUDHL4 cells were plated. The X-axis is the concentration (ng/ml) of VAY736.

FIGS. 4A-4C: Percent specific lysis of SUDHL4 cells in the presence of VAY736 by pNK cells treated with lenalidomide (L) for 24 hr. Unstimulated PBMC cells were treated with Lenalidomide or DMSO for 24 hr, then NK cells isolated from PBMC were added to Calcein AM-labeled SUDHL4 cells for 4 hr in the presence of a dose curve of VAY736, at an E:T of 2:1 (FIG. 4A), 10:1 (FIG. 4B) or 20:1 (FIG. 4C). A total of 7.5e3 SUDHL4 cells were plated. The X-axis is the concentration (ng/ml) of VAY736.

FIGS. 5A-5D: Percent specific lysis of SUDHL4 cells in the presence of VAY736 by NK3.3 cells treated with lenalidomide (L) for 48 hr or 72 hr. NK3.3 cells were treated with lenalidomide or DMSO for 48 hr (FIG. 5B and FIG. 5D) or 72 hr (FIG. 5A and FIG. 5C), then added to Calcein AM-labeled SUDHL4 cells for 2 hr in the presence of a dose curve of VAY736 or isotype control antibody, at an E:T of 20:1 (FIG. 5A and FIG. 5B) or 7:1 (FIG. 5C and FIG. 5D). A total of 7.5e3 SUDHL4 cells were plated. The X-axis is the concentration (ng/ml) of VAY736 or isotype control antibody.

FIGS. 6A-6D: Percent specific lysis of SUDHL4 cells in the presence of VAY736 by NK3.3 cells treated with lenalidomide (L) for 48 hr or 72 hr, with isotype control subtracted. Data corresponds to data shown in FIGS. 5A-5D, with signal from the isotype control subtracted from the VAY736 condition. NK3.3 cells were treated with lenalidomide or DMSO for 48 hr (FIG. 6B and FIG. 6D) or 72 hr (FIG. 6A and FIG. 6C), then added to Calcein AM-labeled SUDHL4 cells for 2 hr in the presence of a dose curve of VAY736 or isotype control antibody, at an E:T of 20:1 (FIG. 6A and FIG. 6B) or 7:1 (FIG. 6C and FIG. 6D). A total of 7.5e3 SUDHL4 cells were plated. The X-axis is the concentration (ng/ml) of VAY736 or isotype control antibody.

FIGS. 7A-7D: Percent specific lysis of SUDHL4 cells in the presence of VAY736 by unstimulated or stimulated PBMC treated with lenalidomide (LEN) for 72 hr. Unstimulated (FIG. 7A and FIG. 7C) or 24 hr IL2 stimulated (FIG. 7B and FIG. 7D) PBMC were treated with lenalidomide or DMSO for 72 hr, then added to Calcein AM-labeled SUDHL4 cells for 3 hr in the presence of a dose curve of VAY736 or isotype control, at an E:T of 3:1. A total of 10e3 SUDHL4 cells were plated. FIG. 7A and FIG. 7B show the isotype control signal subtracted from the signal from VAY736. The X-axis is the concentration (ng/ml) of VAY736 (FIG. 7A and FIG. 7B) or isotype control antibody or VAY736 (FIG. 7C and FIG. 7D). SVC=vehicle control (DMSO).

FIG. 8: Effect of lenalidomide (LEN) on IL2 production in PBMC, with and without CD3+ cells. Unstimulated PBMC, or PBMC with positive CD3+ cell depletion, were treated with Lenalidomide or DMSO for 72 hr, then the supernatants were isolated for IL2 protein analysis, shown in pg/ml.

FIGS. 9A-9D: Percent specific lysis of SUDHL4 cells in the presence of VAY736 by PBMC treated with lenalidomide (LEN) for 72 hr, with and without CD3+ cells. PBMC were treated with Lenalidomide or DMSO for 72 hr, then PBMC (FIG. 9A), pNK cells isolated from the PBMCs (FIG. 9B), or CD3+ depleted PBMC (FIG. 9C) were added to Calcein AM-labeled SUDHL4 cells for 4 hr in the presence of a dose curve of VAY736, at an E:T of 2:1, 6:1 (PBMC), 1:1, 1:3, 1:10 (pNK), or 2:1, 6:1, 15:1 (CD3+ depleted PBMC). A total of 7.5e3 SUDHL4 cells were plated. FIG. 9D is a compilation of data from FIGS. 9A-9C. The X-axis is the concentration (ng/ml) of VAY736.

FIG. 10: A schematic presentation of the treatment regimens of Example 3.

FIG. 11: Percentage change from baseline in blood MRD of the patients treated as in Example 3.

### 7. DETAILED DESCRIPTION

In one aspect, the disclosure provides an anti-BAFFR antibody (e.g., ianalumab) or a binding fragment thereof for use in the treatment of B cell malignancy in a subject in need thereof. For example, the B cell malignancy can be a non-Hodgkin's lymphoma (NHL), for example, chronic lymphocytic leukemia (CLL)/small lymphocytic lymphoma (SLL), follicular lymphoma (FL), mantle cell lymphoma (MCL), diffuse large B-cell lymphoma (DLBCL), Burkitt lymphoma, lymphoplasmacytic lymphoma (Waldenstrom macroglobulinemia), MALT lymphoma (mucosa-associated lymphoid tissue lymphoma) or marginal zone lymphoma (MZL), a leukemia, or multiple myeloma.

The anti-BAFFR antibody or fragment thereof can be administered as monotherapy or administered in combination with one or more additional agents.

In another aspect, the disclosure provides methods of treating subjects having a B cell malignancy with an anti-BAFFR antibody (e.g., ianalumab) or a binding fragment thereof as monotherapy, or in combination with one or more additional agents. In some embodiments, the subject has an NHL, for example DLBCL or MCL, and (i) has failed at least one prior line (and optionally up to five prior lines) of standard of care therapy, e.g., an anti-CD20 therapy such as rituximab and/or (ii) is intolerant to or ineligible for one or more other approved therapies, e.g., autologous stem cell transplant (ASCT) and/or (iii) is a non-responder to a chimeric antigen receptor (CAR) T cell therapy. The NHL can be relapsed and/or refractory.

In another aspect, the disclosure provides combinations comprising an anti-BAFFR antibody (e.g., ianalumab) or a binding fragment thereof and one or more additional agents, for example an immunomodulatory imide drug (IMiD). In some embodiments, a combination comprises ianalumab and lenalidomide.

In another aspect, the disclosure provides for the use of anti-BAFFR antibodies (e.g., ianalumab) and binding fragments thereof and additional agents in the manufacture of medicaments for treating a subject having a B cell malignancy (e.g., a NHL such as DLBCL or MCL). In some embodiments, the subject has an NHL, for example DLBCL or MCL, and (i) has failed at least one prior line (and optionally up to five prior lines) of standard of care therapy, e.g., an anti-CD20 therapy such as rituximab and/or (ii) is intolerant to or ineligible for one or more other approved therapies, e.g., autologous stem cell transplant (ASCT) and/or (iii) is a non-responder to a chimeric antigen receptor (CAR) T cell therapy. The NHL can be relapsed and/or refractory.

In further aspects, the disclosure provides anti-BAFFR antibodies and binding fragments thereof for use in treating a subject with cytokine release syndrome (CRS) or at risk for CRS, methods of reducing the severity of one or more symptoms of CRS in a subject by administering an anti-BAFFR antibody or binding fragment thereof to the subject, and use of anti-BAFFR antibodies and binding fragments thereof in the manufacture of medicaments for treating a subject with CRS or at risk of CRS.

Exemplary anti-BAFFR antibodies and binding fragments are described in Section 7.2. Exemplary anti-BAFFR antibody or binding fragment dosage regimens for use in treating a B cell malignancy are described in Section 7.2. Exemplary additional agents are described in Section 7.3. Exemplary B cell malignancies are described in Section 7.4. Exemplary features of pharmaceutical compositions are described in Section 7.5.

### 7.1. Definitions

As used herein, the following terms are intended to have the following meanings:

A, An, The: As used herein, the term “a”, “an”, “the” and similar terms used in the context of the present disclosure (especially in the context of the claims) are to be construed to cover both the singular and plural unless otherwise indicated herein or clearly contradicted by the context. As such, the terms “a” (or “an”), “one or more”, and “at least one” can be used interchangeably herein.

And/or: The term “and/or” means that each one or both or all of the components or features of a list are possible variants, especially two or more thereof in an alternative or cumulative way.

Additional Agent: For convenience, an agent that is used in combination with an anti-BAFFR antibody or a binding fragment thereof is referred to herein as an “additional” agent.

Antibody: The term “antibody” refers to a protein, e.g., an immunoglobulin chain or fragment thereof, comprising at least one immunoglobulin variable domain sequence. The term “antibody” includes, for example, a monoclonal antibody (including a full length antibody which has an immunoglobulin Fc region). An antibody comprises a full length antibody, or a full length immunoglobulin chain, or an antigen binding or functional fragment of a full length antibody, or a full length immunoglobulin chain. An antibody can also be a multi-specific antibody, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. The term “binding fragment” as used herein refers to a portion of an antibody capable of binding a BAFFR epitope.

Anti-BAFFR antibody: The term “anti-BAFFR antibody or binding fragment thereof” as used herein refers to an antibody, or binding fragment thereof, which comprises a BAFFR binding domain. The binding of the antibody (or binding fragment thereof) to BAFFR inhibits the binding of BAFFR to BAFF and thereby reduces the formation of BAFF/BAFFR complexes, and/or reduce the activation of BAFFR. Suitably, the anti-BAFFR antibody or binding fragment thereof may reduce the formation of BAFF/BAFFR complexes and/or reduce the activation of BAFFR by at least 30%, at least 40%, at least 50%, at least 60%, at least 70%, at least 80%, at least 90%, at least 95%, at least 96%, at least 97%, at least 98%, at least 99%, or more as compared to a suitable control (for example a sample without the presence of an anti-BAFFR antibody or binding fragment thereof). Additionally or alternatively, an anti-BAFFR antibody or binding thereof may dissociate preformed BAFF/BAFFR complexes. In a suitable embodiment antibody or binding fragment thereof may dissociate at least 30%, at least 40%, at least 50%, at least 60%, at least 70%, at least 80%, at least 90%, at least 95%, at least 96%, at least 97%, at least 98%, at least 99%, or more of preformed BAFF/BAFFR complexes. As before, this property may be compared to a suitable control (for example a sample without the presence of an anti-BAFFR antibody or binding fragment thereof).

BAFFR: The term “BAFFR” refers to the B-cell activating factor receptor protein. BAFFR is also known as TNF Receptor Superfamily Member 13C (TNFRSF13C). The human and murine amino acid and nucleic acid sequences can be found in a public database, such as GenBank, UniProt and Swiss-Prot. For example, an amino acid sequence of human BAFFR can be found as UniProt/Swiss-Prot Accession No. Q96RJ3 and a nucleotide sequences encoding human BAFFR can be found at Accession Nos. NM_052945.4. It is expressed predominantly on B-lymphocytes and on a subset of T-cells.

B cell malignancy: As used herein, a B cell malignancy refers to an uncontrolled proliferation of B cells. Examples of B cell malignancies include: non-Hodgkin's lymphomas (NHL), such as diffuse large B-cell lymphoma (DLBCL), small lymphocytic lymphoma (SLL), lymphoplasmacytoid lymphoma, mantle cell lymphoma (MCL), follicular lymphoma, mucosa-associated lymphoid tissue lymphoma (MALT), and Burkitt's lymphoma; precursor B-lymphoblastic leukemia; chronic lymphocytic leukemia; and multiple myeloma.

Chimeric Antigen Receptor: The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. The set of polypeptides can be contiguous or non-contiguous with each other. Where the polypeptides are not contiguous with one another, the set of polypeptides include a dimerization switch that, upon the presence of a dimerization molecule, can couple the polypeptides to one another, e.g., can couple an antigen binding domain to an intracellular signaling domain. CAR molecules are typically administered to a subject by way of administration of immune effector cells (e.g., T cells that are preferably autologous to the subject) engineered to express a CAR molecule.

Combination: The terms “a combination” or “in combination with” is not intended to imply that the therapy or the therapeutic agents must be administered at the same time and/or formulated for delivery together, although these methods of delivery are within the scope described herein. The therapeutic agents in the combination can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. It will further be appreciated that the additional therapeutic agent utilized in this combination may be administered together or separately in different compositions. In general, it is expected that additional therapeutic agents utilized in combination be utilized at levels that do not exceed the levels at which they are utilized individually. In some embodiments, the levels utilized in combination will be lower than those utilized individually.

Drug, active substance, active ingredient, and the like: The terms “drug”, “active substance”, “active ingredient”, “pharmaceutically active ingredient”, “active agent”, “therapeutic agent” or “agent” are to be understood as meaning a compound in free form or in the form of a pharmaceutically acceptable salt.

Effective amount: By the term “effective amount” or “therapeutically effective amount” or “pharmaceutically effective amount”, is meant the amount or quantity of active agent that is sufficient to elicit the required or desired response, or in other words, the amount that is sufficient to elicit an appreciable biological response when administered to a subject. Said amount preferably relates to an amount that is therapeutically or in a broader sense also prophylactically effective against the progression of a disease or disorder as disclosed herein. It is understood that an “effective amount” or a “therapeutically effective amount” can vary from subject to subject, due to variation in metabolism of an agent, age, weight, general condition of the subject, the condition being treated, the severity of the condition being treated, and the judgment of the prescribing physician.

Patient/subject: As used herein, the term “patient” or “subject” are taken to mean a human. Except when noted, the terms “patient” or “subject” are used herein interchangeably.

In need of: As used herein, a subject is “in need of” a treatment if such subject would benefit biologically, medically or in quality of life from such treatment.

Pharmaceutically acceptable: The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable salts: The term “pharmaceutically acceptable salts” can be formed, for example, as acid addition salts, preferably with organic or inorganic acids. Suitable inorganic acids are, for example, halogen acids, such as hydrochloric acid. Suitable organic acids are, e.g., carboxylic acids or sulfonic acids, such as fumaric acid or methanesulfonic acid. For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred. Any reference to the free compound herein is to be understood as referring also to the corresponding salt, as appropriate and expedient. The salts of agents, as described herein, are preferably pharmaceutically acceptable salts; suitable counter-ions forming pharmaceutically acceptable salts are known in the field.

Treat, treating, treatment: As used herein, the term “treat”, “treating” or “treatment” of any disease or disorder refers in one embodiment to ameliorating the disease or disorder (e.g., slowing or arresting or reducing the development of the disease or at least one of the clinical symptoms or pathological features thereof). In another embodiment “treat”, “treating” or “treatment” refers to alleviating or ameliorating at least one physical parameter or pathological features of the disease, e.g. including those, which may not be discernible by the subject. In yet another embodiment, “treat”, “treating” or “treatment” refers to modulating the disease or disorder, either physically, (e.g. stabilization of at least one discernible or non-discernible symptom), physiologically (e.g. stabilization of a physical parameter) or both. In yet another embodiment, “treat”, “treating” or “treatment” refers to preventing or delaying the onset or development or progression of the disease or disorder, or of at least one symptoms or pathological features associated thereof. In yet another embodiment, “treat”, “treating” or “treatment” refers to preventing or delaying progression of the disease to a more advanced stage or a more serious condition. The benefit to a patient to be treated is either statistically significant or at least perceptible to the patient or to the physician. However, it will be appreciated that when a medicament is administered to a patient to treat a disease, the outcome may not always be an effective treatment.

The following abbreviations are used throughout the disclosure.
ADCC Antibody Dependent Cellular Cytotoxicity
AE Adverse Event
ANC Absolute Neutrophil Count
BAFF B-cell Activating Factor
BAFF-R B-cell Activating Factor Receptor
BHLRM Bayesian Hierarchical Logistic Regression Model
BOR Best Overall Response
BTK Bruton's Tyrosine Kinase
BTKi Bruton's Tyrosine Kinase inhibitor
Cl Confidence Interval
CLL Chronic Lymphocytic Leukemia
CR Complete Response
CTCAE Common Terminology Criteria for Adverse Events
DLBCL Diffuse large B-cell lymphoma
DLT Dose Limiting Toxicity
ECOG Eastern Cooperative Oncology Group
EWOC Escalation With Overdose Control
FDA Food and Drug Administration
Hgb Hemoglobin
HIV Human Immunodeficiency Virus
Ig Immunogenicity
IgG Immunoglobulin G
IgG1 Immunoglobulin G1
IUD Intrauterine Device
IUS Intrauterine System
i.v. Intravenous(ly)
mAb Monoclonal antibody
MRD Minimal Residual Disease
MTD Maximum Tolerated Dose
NHL Non-Hodgkin's Lymphoma
NK Natural Killer
NYHA New York Heart Association
ORR Overall Response Rate
PD Pharmacodynamics
PFS Progression-Free Survival
PK Pharmacokinetics
PO Oral administration
PR Partial Response
QD Once Every Day
Q2W Once Every Two Weeks
Q4W Once Every Four Weeks
RD Recommended dose
SAE Serious Adverse Event
SD Stable Disease
TTP Time to Progression
ULN Upper Limit of Normal
WHO World Health Organization

### 7.2. Anti-BAFFR Antibodies

Antibodies against BAFFR (“anti-BAFFR antibodies”) are known from e.g. WO 2010/007082 and include antibodies which are characterized by comprising a VH domain with the amino acid sequence of SEQ ID NO: 1 and a VL domain with the amino acid sequence of SEQ ID NO: 2. The antibody MOR6654 is one such antibody (IgG1 kappa). It has the heavy chain amino acid sequence of SEQ ID NO: 9 and the light chain amino acid sequence of SEQ ID NO: 10. This antibody may be expressed from SEQ ID NOs: 13 and 14, preferably in a host cell which lacks fucosyl-transferase, for example in a mammalian cell line with an inactive FUT8 gene (e.g. FUT8^{-/-}), to provide a functional non-fucosylated anti-BAFFR antibody with enhanced ADCC. This antibody is referred to hereafter as MOR6654B or VAY736, or under its international non-proprietary name ianalumab. Alternative ways to produce non-fucosylated antibodies are known in the art. Amino acid sequences for ianalumab are shown in Table 1, together with nucleic acid sequences encoding ianalumab heavy and light chains.

TABLE 1
ianalumab sequences

| | | SEQ ID |
|---|---|---|
| Portion | Amino acid or Nucleotide Sequence | NO: |
| VH | QVQLQQSGPGLVKPSQTLSLTCAISGDSVSSNSAAWGWIRQSPGRGLEWL | 1 |
| | GRIYYRSKWYNSYAVSVKSRITINPDTSKNQFSLQLNSVTPEDTAVYYCARY | |
| | DWVPKIGVFDSWGQGTLVTVSS | |
| VL | DIVLTQSPATLSLSPGERATLSCRASQFISSSYLSWYQQKPGQAPRLLIYGS | 2 |
| | SSRATGVPARFSGSGSGTDFTLTISSLEPEDFAVYYCQQLYSSPMTFGQGT | |
| | KVEIK | |
| CDR-H1 | GDSVSSNSAAWG | 3 |
| CDR-H2 | RIYYRSKWYNSYAVSVKS | 4 |
| CDR-H3 | YDWWPKIGVFDS | 5 |
| CDR-L1 | RASQFISSSYLS | 6 |
| CDR-L2 | GSSSRAT | 7 |
| CDR-L3 | QQLYSSPMT | 8 |
| Heavy | QVQLQQSGPGLVKPSQTLSLTCAISGDSVSSNSAAWGWIRQSPGRGLEWL | 9 |
| Chain | GRIYYRSKWYNSYAVSVKSRITINPDTSKNQFSLQLNSVTPEDTAVYYCARY | |
| | DWVPKIGVFDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKRVEPKSCDKTHTCPPCPAPELLGGPSVFLFPPKPK | |
| | DTLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVYTL | |
| | PPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGK | |
| Light Chain | DIVLTQSPATLSLSPGERATLSCRASQFISSSYLSWYQQKPGQAPRLLIYGS | 10 |
| | SSRATGVPARFSGSGSGTDFTLTISSLEPEDFAVYYCQQLYSSPMTFGQGT | |
| | KVEIKRTVAAPSVFIFPPSDEQLKSGTASVVCLLNNFYPREAKVQWKVDNAL | |
| | QSGNSQESVTEQDSKDSTYSLSSTLTLSKADYEKHKVYACEVTHQGLSSPV | |
| | TKSFNRGEC | |
| Nucleotide | CAGGTGCAGCTGCAGCAGAGCGGCCCAGGCCTGGTCAAGCCCTCTCAG | 11 |
| sequence | ACCCTGTCACTGACCTGCGCCATTTCAGGCGACAGCGTGAGCAGCAACA | |
| encoding | GCGCCGCCTGGGGCTGGATCAGGCAGAGCCCCGGTAGGGGCCTGGAA | |
| SEQ ID | TGGCTGGGCAGGATCTACTACAGGTCCAAGTGGTACAACAGCTACGCC | |
| NO: 1 | GTGAGCGTGAAGAGCAGGATCACCATCAACCCTGACACCAGCAAGAAC | |
| | CAGTTCTCACTGCAGCTCAACAGCGTGACCCCCGAGGACACCGCCGTG | |
| | TACTACTGCGCCAGATACGACTGGGTGCCCAAGATCGGCGTGTTCGACA | |
| | GCTGGGGCCAGGGCACCCTGGTGACCGTGTCAAGC | |
| Nucleotide | GATATCGTGCTGACACAGAGCCCCGCCACCCTGAGCCTGAGCCCAGGC | 12 |
| sequence | GAGAGGGCCACCCTGTCCTGCAGGGCCAGCCAGTTTATCAGCAGCAGC | |
| encoding | TACCTGTCCTGGTATCAGCAGAAGCCCGGCCAGGCCCCTAGACTGCTG | |
| SEQ ID | ATCTACGGCAGCTCCTCTCGGGCCACCGGCGTGCCCGCCAGGTTCAGC | |
| NO: 2 | GGCAGCGGCTCCGGCACCGACTTCACCCTGACAATCAGCAGCCTGGAG | |
| | CCCGAGGACTTCGCCGTGTACTACTGCCAGCAGCTGTACAGCTCACCCA | 13 |
| | TGACCTTCGGCCAGGGCACCAAGGTGGAGATCAAG | |
| Full length | ATGGCCTGGGTGTGGACCCTGCCCTTCCTGATGGCCGCTGCCCAGTCA | |
| nucleotide | GTGCAGGCCCAGGTGCAGCTGCAGCAGAGCGGCCCAGGCCTGGTCAA | |
| sequence | GCCCTCTCAGACCCTGTCACTGACCTGCGCCATTTCAGGCGACAGCGTG | |
| (including | AGCAGCAACAGCGCCGCCTGGGGCTGGATCAGGCAGAGCCCCGGTAG | |
| leader | GGGCCTGGAATGGCTGGGCAGGATCTACTACAGGTCCAAGTGGTACAA | |
| sequence and | CAGCTACGCCGTGAGCGTGAAGAGCAGGATCACCATCAACCCTGACAC | |
| constant | CAGCAAGAACCAGTTCTCACTGCAGCTCAACAGCGTGACCCCCGAGGA | |
| part) of | CACCGCCGTGTACTACTGCGCCAGATACGACTGGGTGCCCAAGATCGG | |
| heavy | CGTGTTCGACAGCTGGGGCCAGGGCACCCTGGTGACCGTGTCAAGCGC | |
| chain; nt | CAGCACCAAGGGCCCCAGCGTGTTCCCCCTGGCCCCCAGCAGCAAGAG | |
| 1-57 = | CACCAGCGGCGGCACAGCCGCCCTGGGCTGCCTGGTGAAGGACTACTT | |
| leader; nt | CCCCGAGCCCGTGACCGTGTCCTGGAACAGCGGAGCCCTGACCTCCGG | |
| 58-429 = | CGTGCACACCTTCCCCGCCGTGCTGCAGAGCAGCGGCCTGTACAGCCT | |
| VH; nt | GTCCAGCGTGGTGACAGTGCCCAGCAGCAGCCTGGGCACCCAGACCTA | |
| 430-1419 = | CATCTGCAACGTGAACCACAAGCCCAGCAACACCAAGGTGGACAAGAGA | |
| constant | GTGGAGCCCAAGAGCTGCGACAAGACCCACACCTGCCCCCCCTGCCCA | |
| region | GCCCCAGAGCTGCTGGGCGGACCCTCCGTGTTCCTGTTCCCCCCCAAG | |
| (hIgG1) | CCCAAGGACACCCTGATGATCAGCAGGACCCCCGAGGTGACCTGCGTG | |
| | GTGGTGGACGTGAGCCACGAGGACCCAGAGGTGAAGTTCAACTGGTAC | |
| | GTGGACGGCGTGGAGGTGCACAACGCCAAGACCAAGCCCAGAGAGGA | |
| | GCAGTACAACAGCACCTACAGGGTGGTGTCCGTGCTGACCGTGCTGCA | |
| | CCAGGACTGGCTGAACGGCAAGGAATACAAGTGCAAGGTCTCCAACAA | |
| | GGCCCTGCCAGCCCCCATCGAAAAGACCATCAGCAAGGCCAAGGGCCA | |
| | GCCACGGGAGCCCCAGGTGTACACCCTGCCCCCCTCCCGGGAGGAGAT | |
| | GACCAAGAACCAGGTGTCCCTGACCTGTCTGGTGAAGGGCTTCTACCCC | |
| | AGCGACATCGCCGTGGAGTGGGAGAGCAACGGCCAGCCCGAGAACAAC | |
| | TACAAGACCACCCCCCCAGTGCTGGACAGCGACGGCAGCTTCTTCCTGT | |
| | ACAGCAAGCTGACCGTGGACAAGTCCAGGTGGCAGCAGGGCAACGTGT | |
| | TCAGCTGCAGCGTGATGCACGAGGCCCTGCACAACCACTACACCCAGA | |
| | AGAGCCTGAGCCTGTCCCCCGGCAAG | |
| Full length | ATGAGCGTGCTGACCCAGGTGCTGGCTCTGCTGCTGCTGTGGCTGACC | 14 |
| nucleotide | GGCACCAGATGCGATATCGTGCTGACACAGAGCCCCGCCACCCTGAGC | |
| sequence | CTGAGCCCAGGCGAGAGGGCCACCCTGTCCTGCAGGGCCAGCCAGTTT | |
| (including | ATCAGCAGCAGCTACCTGTCCTGGTATCAGCAGAAGCCCGGCCAGGCC | |
| leader | CCTAGACTGCTGATCTACGGCAGCTCCTCTCGGGCCACCGGCGTGCCC | |
| sequence and | GCCAGGTTCAGCGGCAGCGGCTCCGGCACCGACTTCACCCTGACAATC | |
| constant | AGCAGCCTGGAGCCCGAGGACTTCGCCGTGTACTACTGCCAGCAGCTG | |
| part) of | TACAGCTCACCCATGACCTTCGGCCAGGGCACCAAGGTGGAGATCAAG | |
| light chain; | CGTACGGTGGCCGCTCCCAGCGTGTTCATCTTCCCCCCCAGCGACGAG | |
| nt 1-60 = | CAGCTGAAGAGCGGCACCGCCAGCGTGGTGTGCCTGCTGAACAACTTC | |
| leader; nt | TACCCCCGGGAGGCCAAGGTGCAGTGGAAGGTGGACAACGCCCTGCAG | |
| 61-384 = | AGCGGCAACAGCCAGGAGAGCGTCACCGAGCAGGACAGCAAGGACTCC | |
| VL; nt | ACCTACAGCCTGAGCAGCACCCTGACCCTGAGCAAGGCCGACTACGAG | |
| 385-705 = | AAGCATAAGGTGTACGCCTGCGAGGTGACCCACCAGGGCCTGTCCAGC | |
| constant | CCCGTGACCAAGAGCTTCAACAGGGGCGAGTGC | |
| region | | |
| (hkappa) | | |

In some embodiments, the anti-BAFFR antibody or binding fragment thereof comprises a heavy chain variable region comprising three CDRs having sequences of SEQ ID NO: 3, SEQ ID NO: 4, and SEQ ID NO: 5, respectively, and a light chain variable region comprising three CDRs having sequences of SEQ ID NO: 6, SEQ ID NO: 7, and SEQ ID NO: 8, respectively. In a preferred embodiment, the anti-BAFFR antibody or binding fragment thereof comprises a heavy chain variable region consisting of the sequence SEQ ID NO: 1 and a light chain variable region consisting of the sequence SEQ ID NO: 2. In a more preferred embodiment, the anti-BAFFR antibody or binding fragment thereof is ianalumab or binding fragment thereof.

In some embodiments, the anti-BAFFR antibody (e.g., ianalumab) or binding fragment thereof is administered at a dose of 0.1 mg/kg to 20 mg/kg (e.g., 1 mg/kg to 10 mg/kg, 5 mg/kg to 15 mg/kg, or 10 mg/kg to 20 mg/kg). In some embodiments, the anti-BAFFR antibody (e.g., ianalumab) or binding fragment thereof is administered at a dose of 1 mg/kg to 10 mg/kg. In some embodiments, the anti-BAFFR antibody (e.g., ianalumab) or binding fragment thereof is administered at a dose of 10 mg/kg to 20 mg/kg. In some embodiments, the anti-BAFFR antibody (e.g., ianalumab) or binding fragment thereof is administered at a dose of about 1 mg/kg. In some embodiments, the anti-BAFFR antibody (e.g., ianalumab) or binding fragment thereof is administered at a dose of about 3 mg/kg. In some embodiments, the anti-BAFFR antibody (e.g., ianalumab) or binding fragment thereof is administered at a dose of about 6 mg/kg. In some embodiments, the anti-BAFFR antibody (e.g., ianalumab) or binding fragment thereof is administered at a dose of about 9 mg/kg. In some embodiments, the anti-BAFFR antibody (e.g., ianalumab) or binding fragment thereof is administered at a dose of about 12 mg/kg. In some embodiments, the anti-BAFFR antibody (e.g., ianalumab) or binding fragment thereof is administered at a dose of about 20 mg/kg.

The anti-BAFFR antibody (e.g., ianalumab) or binding fragment thereof can in some embodiments be administered to a subject once every two weeks. For example, the anti-BAFFR antibody (e.g., ianalumab) can be administered to a subject on days 1 and 15 of a 28-day dosing cycle. An administration window (e.g., +/−3 days, +/−2 days, or +/−1 day) around the biweekly date can be used.

Alternatively, the anti-BAFFR antibody (e.g., ianalumab) or binding fragment thereof can administered more frequently or less frequently, for example, once a week (e.g., on days 1, 8, 15, and 22 of a 28-day cycle) or once every 4 weeks (e.g., on day 1 of a 28-day cycle). An administration window (e.g., +/−3 days, +/−2 days, or +/−1 day) around the weekly or once every 4 week date can be used.

In an embodiment, the anti-BAFFR antibody or a binding fragment thereof is administered once every two weeks (+/−3 days) at a dose of about 3 mg/kg. In a preferred embodiment, ianalumab or binding fragment thereof is administered once every two weeks (+/−3 days) at a dose of about 3 mg/kg.

In an embodiment, the anti-BAFFR antibody or a binding fragment thereof is administered once every four weeks (+/−3 days) at a dose of about 9 mg/kg. In a preferred embodiment, ianalumab or binding fragment thereof is administered once every four weeks (+/−3 days) at a dose of about 9 mg/kg.

The anti-BAFFR antibody (e.g., ianalumab) or binding fragment thereof can be administered for multiple cycles, e.g., multiple 28-day cycles. In some embodiments, the anti-BAFFR antibody (e.g., ianalumab) or binding fragment thereof is administered for 12 or more cycles.

The anti-BAFFR antibody (e.g., ianalumab) or binding fragment thereof is preferably administered to a subject by intravenous administration.

### 7.3. Additional Agents

An anti-BAFFR antibody or binding fragment thereof (e.g., ianalumab) can be administered in combination with one or more additional agents, for example an immunomodulatory imide drug (IMiD), for example, lenalidomide or a pharmaceutically acceptable salt thereof, and/or one or more supportive agents.

In some aspects, an anti-BAFFR antibody (e.g., ianalumab) is administered in combination with an immunomodulatory imide drug (IMiD), for example, lenalidomide or a pharmaceutically acceptable salt thereof. Lenalidomide is marketed as REVLIMID® (Celgene) and is available in 2.5 mg, 5 mg, 15 mg, 20 mg, and 25 mg dosage forms. Lenalidomide can be administered daily (e.g., for days 1-21 of a 28 cycle), for example at a daily dose of 2.5 mg to 25 mg (e.g., 2.5 mg, 5 mg, 15 mg, 20 mg, or 25 mg). In some embodiments, lenalidomide is administered to a subject for a maximum of 12 cycles, after which treatment with an anti-BAFFR antibody (e.g., ianalumab) can be continued as monotherapy.

Other IMiDs include thalidomide, pomalidomide, and iberdomide. Lenalidomide, pomalidomide, and iberdomide are thalidomide analogues.

Supportive agents that can be used include anti-emetic agents (e.g., phenothiazines, such as prochlorperazine and chlorpromazine, 5-HT3-receptor-selective antagonists such as ondansetron, granisetron, and tropisetron) and anti-diarrheal agents (e.g., loperamide).

An anti-BAFFR antibody or binding fragment thereof and an additional agent can be administered simultaneously, in the same or in separate compositions, or sequentially. For sequential administration, the anti-BAFFR antibody or binding fragment thereof can be administered first, and the additional agent can be administered second, or the order of administration can be reversed.

### 7.4. B Cell Malignancies and Patient Populations

The anti-BAFFR antibodies (e.g., ianalumab) and binding fragments thereof can be used to treat a subject having a B cell malignancy.

In some embodiments, the B cell malignancy is a hematological cancer.

In some embodiments, the B cell malignancy is a malignant lymphoproliferative condition.

In some embodiments, the B cell malignancy is a plasma cell dyscrasia.

In some embodiments, the B cell malignancy is an acute leukemia. In some embodiments, the B cell malignancy is B cell acute lymphocytic leukemia (also known as B cell acute lymphoblastic leukaemia or B cell acute lymphoid leukemia) (ALL or B-ALL), e.g., relapsed and/or refractory B-ALL.

In some embodiments, the B cell malignancy is a non-Hodgkin's lymphoma (NHL), for example, chronic lymphocytic leukemia (CLL)/small lymphocytic lymphoma (SLL), follicular lymphoma (FL), mantle cell lymphoma (MCL), diffuse large B-cell lymphoma (DLBCL), Burkitt lymphoma, lymphoplasmacytic lymphoma (Waldenstrom macroglobulinemia), MALT lymphoma (mucosa-associated lymphoid tissue lymphoma) marginal zone lymphoma (MZL) (e.g., extranodal marginal zone lymphoma (EMZL) or nodal marginal zone B-cell lymphoma (NZML)).

In some embodiments, the B cell malignancy is a relapsed and/or refractory non-Hodgkin's lymphoma (NHL).

In some embodiments, the B cell malignancy is chronic lymphocytic leukemia (CLL)/small lymphocytic lymphoma (SLL), e.g., relapsed and/or refractory CLL/SLL.

In some embodiments, the B cell malignancy is follicular lymphoma (FL), e.g., relapsed and/or refractory FL. In some embodiments, the FL is small cell FL. In other embodiments, the FL is large cell FL.

In some embodiments, the B cell malignancy is mantle cell lymphoma (MCL), e.g., relapsed and/or refractory MCL.

In some embodiments, the B cell malignancy is diffuse large B-cell lymphoma (DLBCL), e.g., relapsed and/or refractory DLBCL.

In some embodiments, the B cell malignancy is DLBCL, e.g., relapsed and/or refractory DLBCL, and the anti-BAFFR antibody or a binding fragment thereof is administered once every four weeks (+/−3 days) at a dose of about 9 mg/kg. In a preferred embodiment, the B cell malignancy is DLBCL, e.g., relapsed and/or refractory DLBCL, and ianalumab or binding fragment thereof is administered once every four weeks (+/−3 days) at a dose of about 9 mg/kg.

In some embodiments, the B cell malignancy is DLBCL, e.g., relapsed and/or refractory DLBCL, and the anti-BAFFR antibody or a binding fragment thereof is administered once every two weeks (+/−3 days) at a dose of about 3 mg/kg. In a preferred embodiment, the B cell malignancy is DLBCL, e.g., relapsed and/or refractory DLBCL, and ianalumab or binding fragment thereof is administered once every two weeks (+/−3 days) at a dose of about 3 mg/kg.

In some embodiments, the B cell malignancy is DLBCL, e.g., relapsed and/or refractory DLBCL, and the anti-BAFFR antibody or a binding fragment thereof is administered in combination with an IMiD, where the anti-BAFFR antibody or a binding fragment thereof is administered once every four weeks (+/−3 days) at a dose of about 9 mg/kg. In a preferred embodiment, the B cell malignancy is DLBCL, e.g., relapsed and/or refractory DLBCL, and ianalumab or binding fragment thereof is administered in combination with an IMiD, where the ianalumab or binding fragment thereof is administered once every four weeks (+/−3 days) at a dose of about 9 mg/kg. In some embodiments, the IMiD is lenalidomide or a pharmaceutically acceptable salt thereof. In other embodiments, the IMiD is thalidomide or a pharmaceutically acceptable salt thereof. In other embodiments, the IMiD is pomalidomide or a pharmaceutically acceptable salt thereof. In other embodiments, the IMiD is iberdomide or a pharmaceutically acceptable salt thereof.

In some embodiments, the B cell malignancy is DLBCL, e.g., relapsed and/or refractory DLBCL, and the anti-BAFFR antibody or a binding fragment thereof is administered in combination with an IMiD, where the anti-BAFFR antibody or a binding fragment thereof is administered once every two weeks (+/−3 days) at a dose of about 3 mg/kg. In a preferred embodiment, the B cell malignancy is DLBCL, e.g., relapsed and/or refractory DLBCL, and ianalumab or binding fragment thereof is administered in combination with an IMiD, where the ianalumab or binding fragment thereof is administered once every two weeks (+/−3 days) at a dose of about 3 mg/kg. In some embodiments, the IMiD is lenalidomide or a pharmaceutically acceptable salt thereof. In other embodiments, the IMiD is thalidomide or a pharmaceutically acceptable salt thereof. In other embodiments, the IMiD is pomalidomide or a pharmaceutically acceptable salt thereof. In other embodiments, the IMiD is iberdomide or a pharmaceutically acceptable salt thereof.

In some embodiments, the B cell malignancy is Burkitt lymphoma.

In some embodiments, the B cell malignancy is lymphoplasmacytic lymphoma (Waldenstrom macroglobulinemia).

In some embodiments, the B cell malignancy is MALT lymphoma (mucosa-associated lymphoid tissue lymphoma).

In some embodiments, the B cell malignancy is marginal zone lymphoma (MZL).

In some embodiments, the B cell malignancy is extranodal marginal zone lymphoma (EMZL).

In some embodiments, the B cell malignancy is nodal marginal zone B-cell lymphoma (NZML).

In some embodiments, the B cell malignancy is splenic marginal zone B-cell lymphoma (SMZL).

In some embodiments, the B cell malignancy is a Hodgkin's lymphoma.

In some embodiments, the B cell malignancy is multiple myeloma.

In some embodiments, the B cell malignancy is hairy cell leukemia.

In some embodiments, the B cell malignancy is primary effusion lymphoma.

In some embodiments, the B cell malignancy is B cell prolymphocytic leukemia.

In some embodiments, the B cell malignancy is plasmablastic lymphoma.

In some embodiments, the B cell malignancy is follicle center lymphoma.

In some embodiments, the B cell malignancy is precursor B-lymphoblastic leukemia.

In some embodiments, the B cell malignancy is high-grade B-cell lymphoma.

In some embodiments, the B cell malignancy is primary mediastinal large B-cell lymphoma.

Certain aspects of the foregoing embodiments relate to subjects having an NHL and who (i) have failed at least one prior line (and optionally up to five prior lines) of standard of care therapy, e.g., an anti-CD20 therapy such as rituximab and/or (ii) is intolerant to or ineligible for one or more other approved therapies, e.g., autologous stem cell transplant (ASCT) and/or (iii) is a non-responder to a chimeric antigen receptor (CAR) T cell therapy. The NHL can be chronic lymphocytic leukemia (CLL)/small lymphocytic lymphoma (SLL), follicular lymphoma (FL), mantle cell lymphoma (MCL), diffuse large B-cell lymphoma (DLBCL), Burkitt lymphoma, lymphoplasmacytic lymphoma (Waldenstrom macroglobulinemia), MALT lymphoma (mucosa-associated lymphoid tissue lymphoma) marginal zone lymphoma (MZL) (e.g., extranodal marginal zone lymphoma (EMZL) or nodal marginal zone B-cell lymphoma (NZML)). In some embodiments, the NHL can relapsed and/or refractory, such as relapsed and/or refractory DLBCL or MCL.

Thus, in certain aspects, a subject having an NHL to whom an anti-BAFFR antibody (e.g., ianalumab) or a binding fragment or a combination comprising an anti-BAFFR antibody (e.g., ianalumab) or binding fragment (e.g., a combination comprising any anti-BAFFR antibody and IMiD such as lenalidomide) is administered has failed at least one prior line of standard of care therapy and optionally up to five standard of care therapies. In various embodiments, the subject has failed one, two, three, four or five standard of care therapies. Exemplary standard of care therapies for B cell malignancies include anti-CD20 therapies such as rituximab.

In further aspects, a subject having an NHL to whom an anti-BAFFR antibody (e.g., ianalumab) or a binding fragment or a combination comprising an anti-BAFFR antibody (e.g., ianalumab) or binding fragment (e.g., a combination comprising any anti-BAFFR antibody and IMiD such as lenalidomide) is administered is intolerant to or ineligible for one or more other approved therapies, e.g., autologous stem cell transplant (ASCT).

In yet further aspects, a subject having an NHL to whom an anti-BAFFR antibody (e.g., ianalumab) or a binding fragment or a combination comprising an anti-BAFFR antibody (e.g., ianalumab) or binding fragment (e.g., a combination comprising any anti-BAFFR antibody and IMiD such as lenalidomide) is administered is a non-responder to chimeric antigen receptor (CAR) T cell therapy composition (“CAR composition”), e.g., an anti-CD19 CAR composition. In certain embodiments, the CAR composition comprises CTL019. In other embodiments, the CAR composition has the USAN or INN designation tisagenlecleucel. Tisagenlecleucel is marketed as KYMRIAH®. See, e.g., KYMRIAH® prescribing information, available at www.pharma.us.novartis.com/sites/www.pharma.us.novartis.com/files/kymriah.pdf. In other embodiments, the CAR composition has the USAN or INN designation axicabtagene ciloleucel. Axicabtagene ciloleucel is marketed as YESCARTA®. See, e.g., YESCARTA® prescribing information, available at www.yescarta.com/files/yescarta-pi.pdf. In other aspects, the CAR composition has the USAN designation brexucabtagene autoleucel. Brexucabtagene autoleucel is marketed as TECARTUS™. See, e.g., TECARTUS™ prescribing information, available at www.gilead.com/-/media/files/pdfs/medicines/oncology/tecartus/tecartus-pi.pdf. In yet other embodiments, the CAR composition has the USAN or INN designation lisocabtagene maraleucel. Lisocabtagene maraleucel is marketed as BREYANZI®. See, e.g., BREYANZI® prescribing information, available at packageinserts.bms.com/pi/pi_breyanzi.pdf.

### 7.5. Pharmaceutical Compositions

The anti-BAFFR antibodies and fragments thereof and additional agents can be formulated as pharmaceutical compositions containing one or more pharmaceutically acceptable excipients or carriers. To prepare pharmaceutical or sterile compositions, an agent preparation can be combined with one or more pharmaceutically acceptable excipients and/or carriers. For combination therapies, the anti-BAFFR antibody or fragment thereof and an additional agent(s) are typically formulated as separate pharmaceutical compositions. Each can be provided, for example, in a single dose or multiple dose container.

For example, formulations of anti-BAFFR antibodies and additional agents can be prepared by mixing the agents with physiologically acceptable carriers, excipients, or stabilizers in the form of, e.g., lyophilized powders, slurries, aqueous solutions, lotions, or suspensions (see, e.g., Hardman et al., 2001, Goodman and Gilman's The Pharmacological Basis of Therapeutics, McGraw-Hill, New York, N.Y.; Gennaro, 2000, Remington: The Science and Practice of Pharmacy, Lippincott, Williams, and Wilkins, New York, N.Y.; Avis, et al. (eds.), 1993, Pharmaceutical Dosage Forms: General Medications, Marcel Dekker, NY; Lieberman, et al. (eds.), 1990, Pharmaceutical Dosage Forms: Tablets, Marcel Dekker, NY; Lieberman, et al. (eds.), 1990, Pharmaceutical Dosage Forms: Disperse Systems, Marcel Dekker, NY; Weiner and Kotkoskie, 2000, Excipient Toxicity and Safety, Marcel Dekker, Inc., New York, N.Y.). Anti-BAFFR antibody formulations are described in WO 2012/076670 and WO 2013/186700, the contents of which are incorporated herein by reference in their entireties.

### 8. EXAMPLES

Ianalumab (VAY736) is a human IgG1/K mAb designed to target human BAFF-R and to competitively inhibit binding of BAFF to BAFF-R, thereby blocking BAFF-R-mediated signaling in B cells. In addition, ianalumab was engineered to effectively eliminate B cells from circulation in vivo by ADCC. Thus, ianalumab eliminates BAFF-R⁺ mature and immature B cells via dual mechanisms: (1) antibody-dependent cytotoxicity (ADCC) and (2) induction of B cell apoptosis by blocking BAFF:BAFF-R interaction and downstream survival pathway in B cells.

Example 1 below shows that ianalumab reduces tumor growth in a DLBCL model. Example 2 below shows that lenalidomide enhances ianalumab-induced ADCC of a DLBCL cell line in vitro. Example 3 below presents preliminary safety and activity data from a clinical trial of ianalumab in combination with ibrutinib in subjects having relapsed/refractory CLL. Taken together, the data supports the use of VAY736 in patients having NHL, as a single agent, or as a “backbone” agent in combination with additional agents, as described in Example 4.

Examples 4-5 below show that the anti-BAFFR antibody VAY736 is capable of depleting healthy B cells in vivo in both mouse and cynomolgus monkey. Without being bound by theory, it is believed that cytokine release by normal B cells is an important driver in CRS, and it is believed that depleting normal B cells in a subject with VAY736 can reduce the severity of CRS experienced by a subject.

### 8.1. Example 1: VAY736 Slows Tumor Growth in DLBCL Model

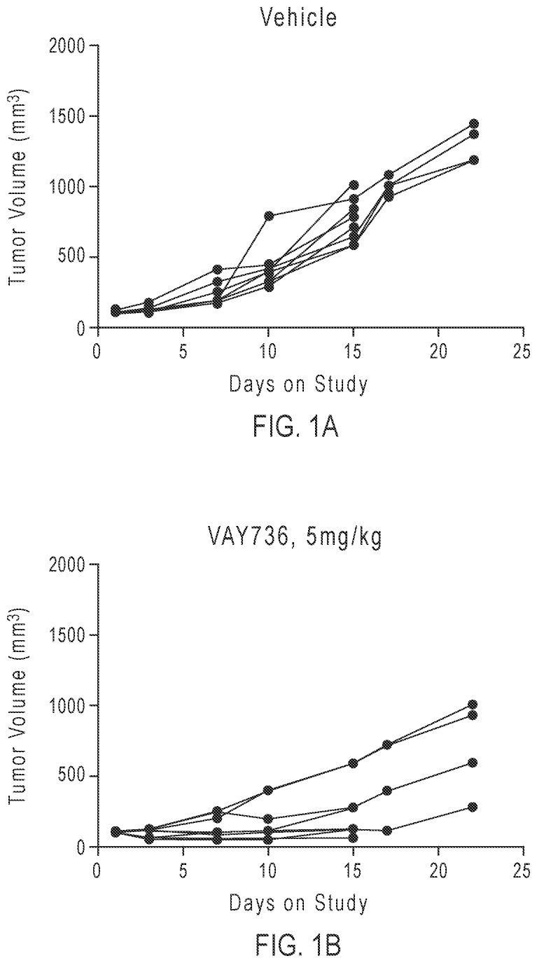

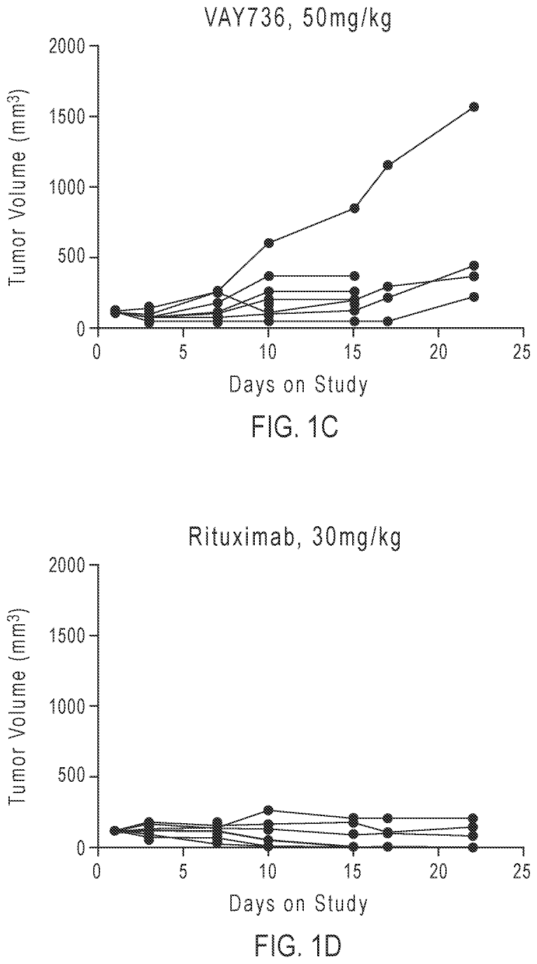

A study was performed to assess the effect of VAY736 in an in vivo DLBCL model. Briefly, the DLBCL cell line SUDHL4 was implanted subcutaneously into SCID mice, which were then treated weekly with 5 mg/kg or 50 mg/kg VAY736 intravenously. Vehicle and rituximab were used as controls. As shown in FIGS. 1A-1C, VAY736 treatment significantly slowed tumor growth in the model compared to the vehicle control, as assessed by tumor volume measurements collected over time.

### 8.2. Example 2: VAY736 in Combination with Lenalidomide Enhances VAY736-Induced ADCC In Vitro

The studies described in this Example were carried out to determine whether lenalidomide can increase the ADCC potential of various effector lines with VAY736 on a NHL cell line (SUDHL4, which is a DLBCL cell line) in vitro. Several different effector cells were utilized, including PBMC, pNK cells and the NK3.3 cell line.

### 8.2.1. Materials and Methods

### 8.2.1.1. Effector Cell Preparation

### 8.2.1.1.1. NK3.3 Cells

NK3.3 cells were collected, washed 1×, and incubated in starvation media with or without 3 uM lenalidomide. Cells were incubated for 48 hrs or 72 hrs, collected, spun down, and resuspended in assay buffer. 100 μl of NK3.3 cells at the designated E:T ratios were transferred to a 96 well plate with SUDHL4 cells and antibodies. The co-culture was incubated for 2 hrs.

### 8.2.1.1.2. PBMC/pNK Cells

Frozen PBMC were thawed, or fresh PBMCs were isolated. If T cells were depleted, PBMC were used for a positive depletion of CD3 using CD3 beads (Miltenyi #130-050-101). Cells were washed 1×, and incubated in unstimulated media (phenol red free/glutamax/hepes/anti-penicillin anti-streptomycin/FBS) with or without 3 μM, 1 μM or 10 μM Lenalidomide. Cells were incubated for 24 hrs or 72 hrs. The supernatant was taken and frozen for IL2 analysis. In one study, for the 72 hr culture, 100 pg/ml IL2 was added to the culture for the last 24 hrs. Primary NK cells were isolated the day of the co-culture setup. 100 μl of PBMC or pNK cells at the designated E:T ratios were transferred to the 96 well plate with the SUDHL4 cells and antibodies. The co-culture was incubated for 3 hrs or 4 hrs.

### 8.2.1.2. Co-Culture Setup and ADCC Assay Readout

A total of 10e6 or 4e6 SUDHL4 cells were incubated with Calcein AM for 60 minutes. Cells were then washed 2× with 30 ml of media. Cells were counted, and resuspended to 0.15 e6/ml or 0.2 e6/ml, and in one study 40 ng/ml of IL2 was added. A total of 50 μl cells were then transferred to a 96 well plate. VAY736 or an afucosylated isotype control were then diluted, and 50 μl were transferred to cells and incubated for 20 min at RT. Effector cells were then added to the 96 well plate. The plate was then spun in a centrifuge without the brake at 200×g for 4 min, and the co-culture was incubated for 2 hrs, 3 hrs or 4 hrs. The plate was then spun without the brake at 1500 RPM for 4 min. 100 μl of the supernatant was moved to black 96 well plates (Corning, #3904) and fluorescence was recorded with a PerkinElmer Envision plate reader.

### 8.2.2. Results

In vitro ADCC assays using the SUDHL4 cell line and various effector cells, including PBMC, pNK cells, and the NK3.3 cell line, demonstrated potent ADCC in the presence of VAY736. The data generally demonstrated a dose-dependent VAY736-dependent specific lysis of SUDHL4 cells. Addition of lenalidomide generally demonstrated enhanced VAY736-dependent percent specific lysis of SUDHL4 cells, with some cultures and treatment conditions resulting in larger enhancements compared to others.

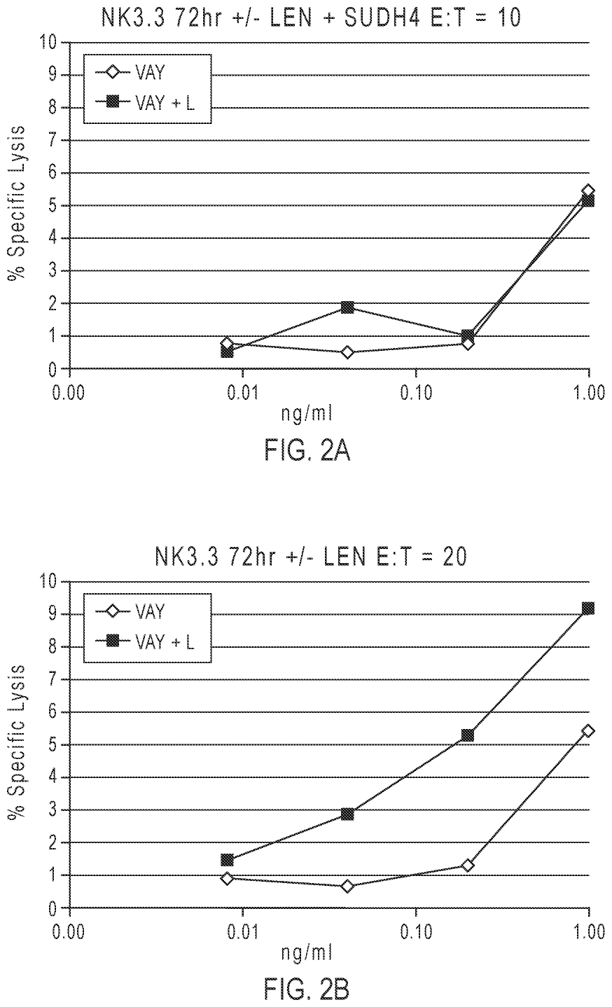

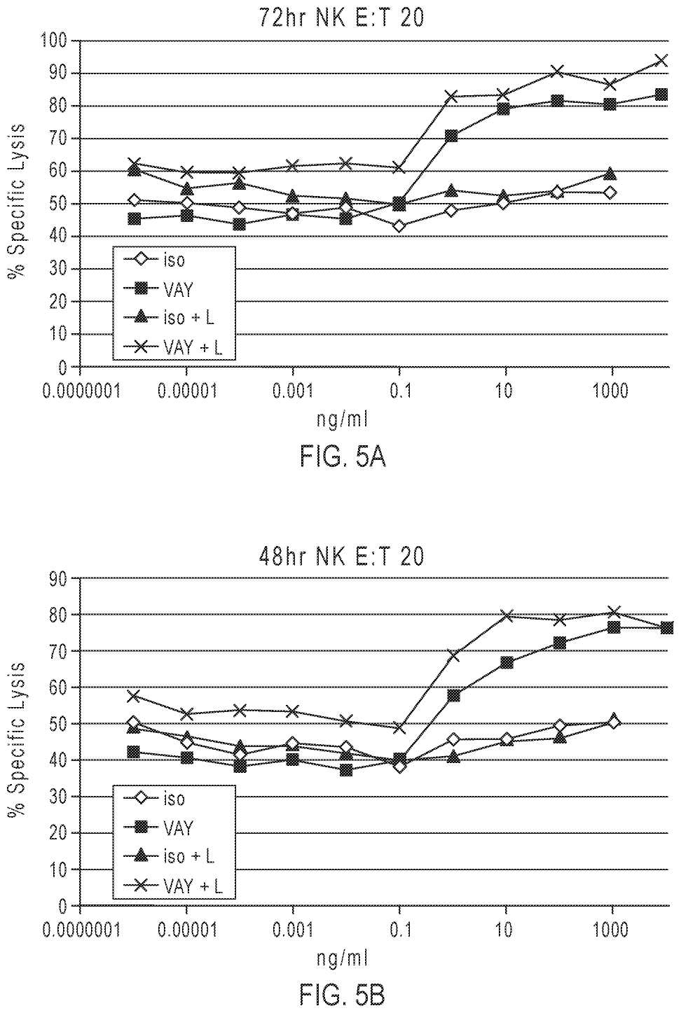

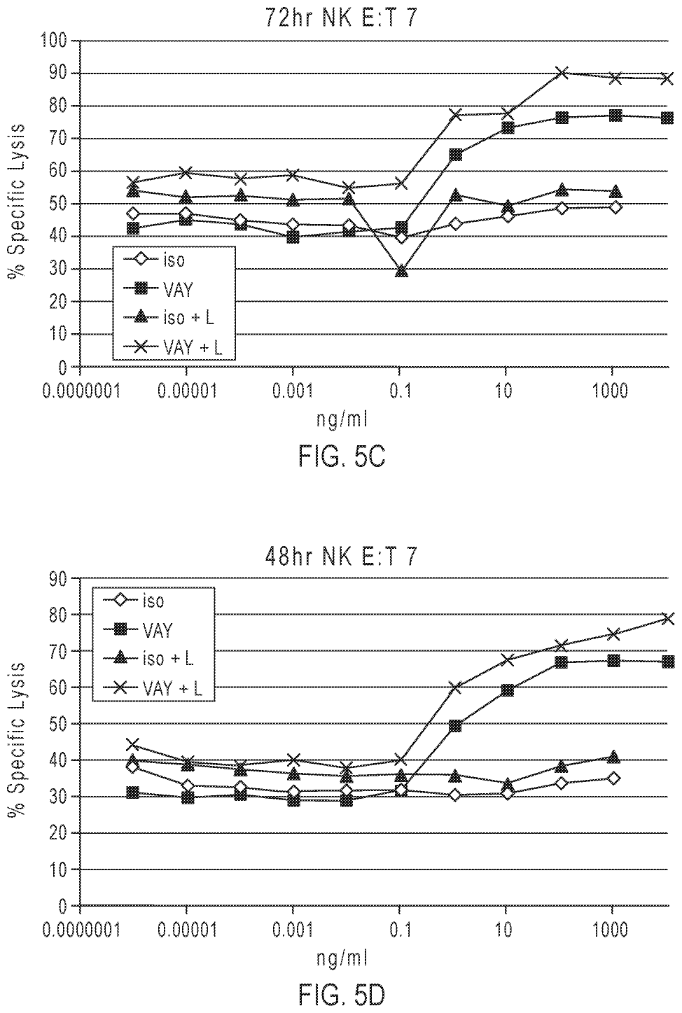

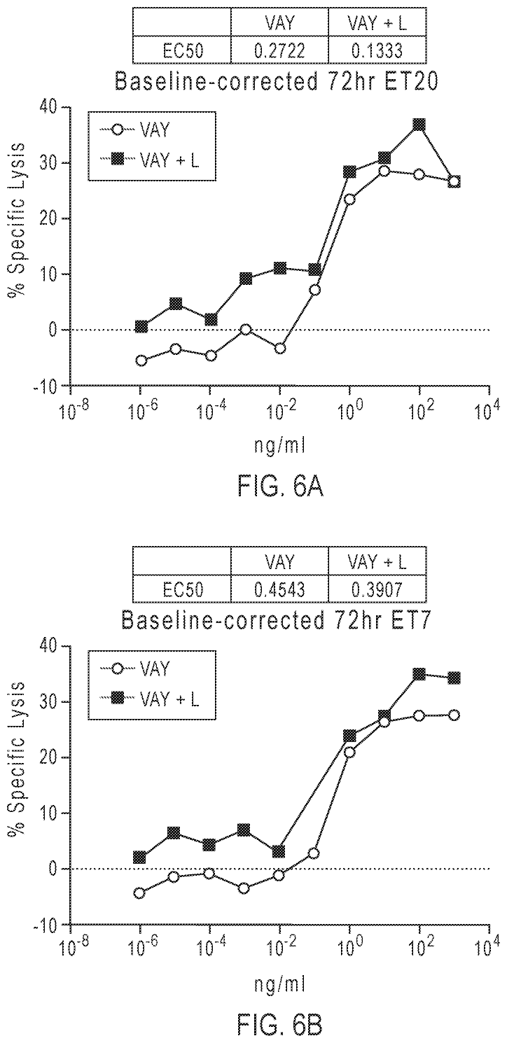

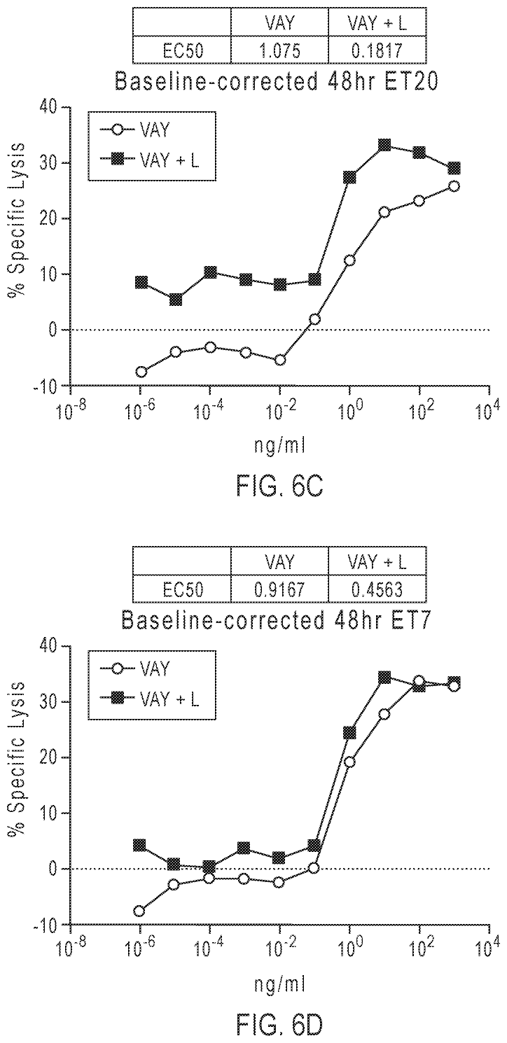

FIGS. 2A-2B, 5A-5D and 6A-6D show results where NK3.3 cells were included as the effector cells. Increases in VAY736-induced ADCC were observed with addition of lenalidomide, which was more prominent with 72 hr pretreatment with lenalidomide at an E:T of 20:1, as shown in FIG. 2A-2B. Slight increases in ADCC with lenalidomide pretreatment were observed at E:T ratios of 20:1 and 7:1 at 72 hr and 48 hr lenalidomide pretreatment (FIGS. 5A-5D and 6A-6D).

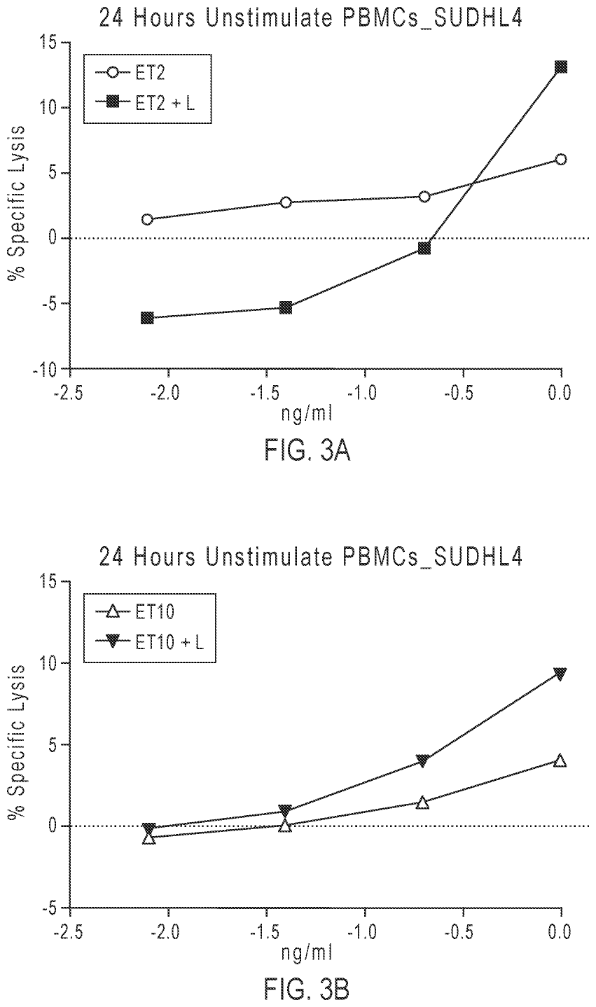

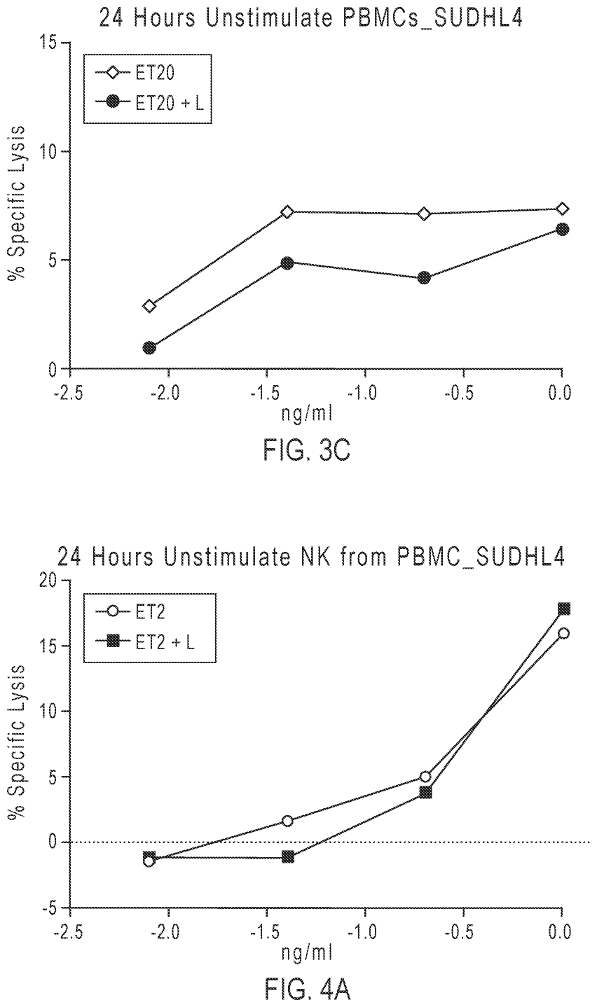

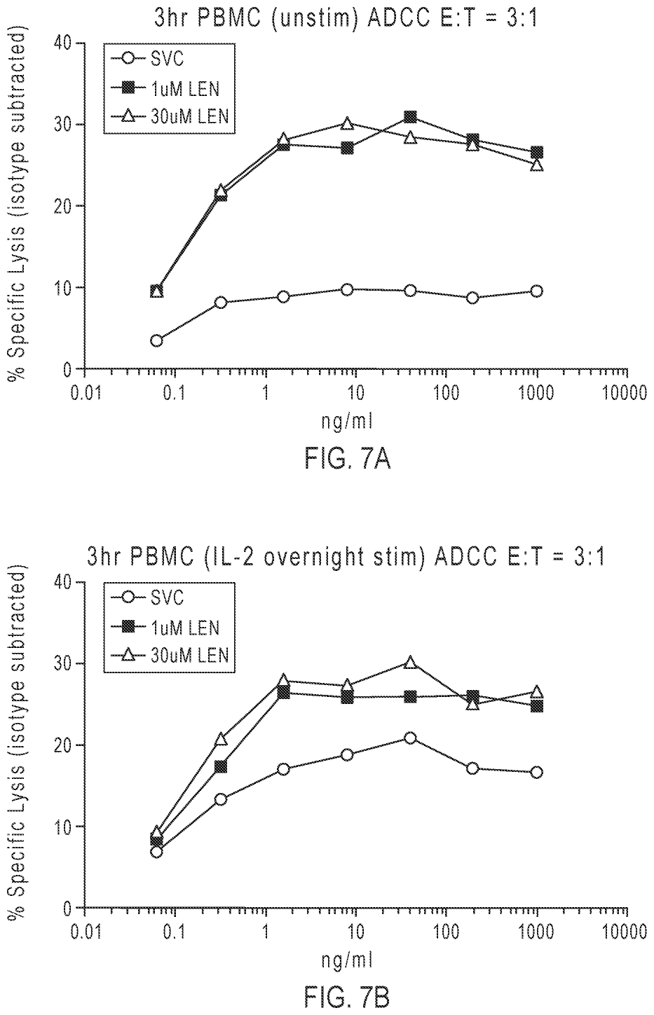

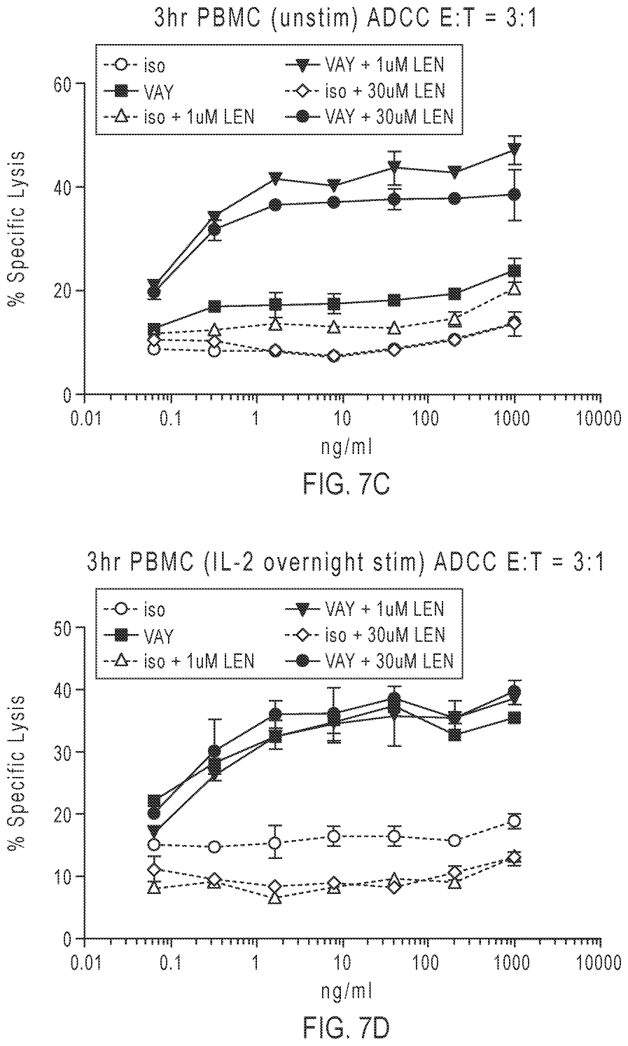

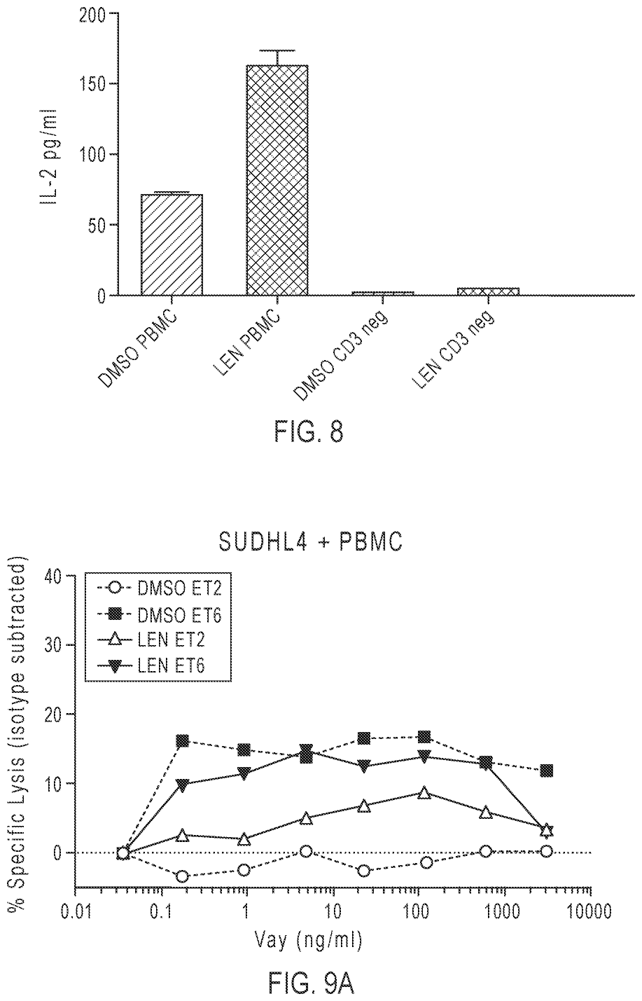

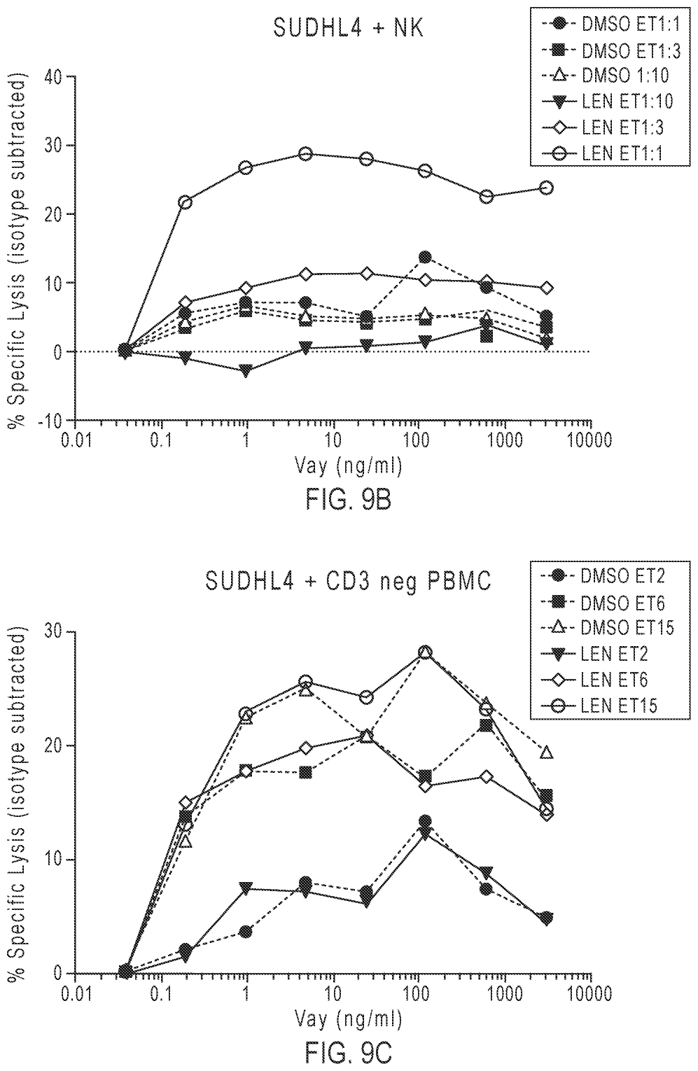

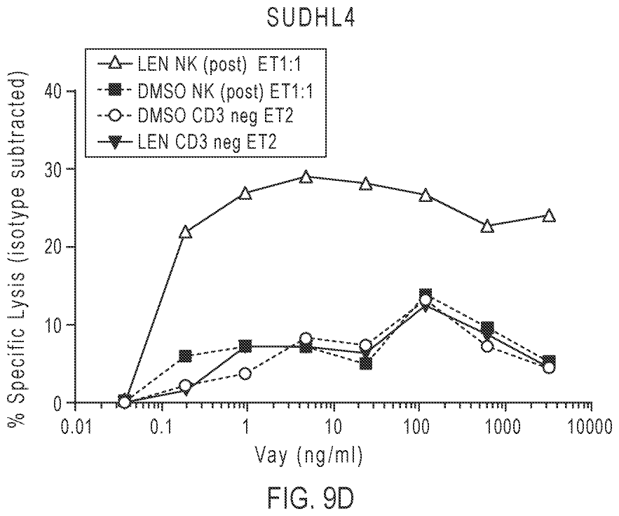

FIGS. 3A-3C, 7A-7D, and 9A, 9C, and 9D show results where PBMC were included as the effector cells. Pretreatment for 24 hrs of PBMC with lenalidomide showed an enhanced VAY736-induced ADCC at an E:T ratio of 10:1, where 2:1 only showed an enhancement at the highest concentration and only a slight enhancement was seen at an E:T of 20:1 (FIG. 3A-3C). FIG. 7A-7D demonstrates that lenalidomide increased VAY736-induced ADCC at 3:1, and this increase was largely dose-independent and hampered with IL2 stimulation of PBMC. Improvements in VAY736-induced ADCC with lenalidomide were less pronounced at 6:1 with 72 hr lenalidomide treatment, but was evident at 2:1 (FIG. 9A). This enhancement was dependent on the presence of CD3+ T cells (FIGS. 9A, 9C, and 9D), likely from the IL2 produced by lenalidomide treatment by T cells (FIG. 8).

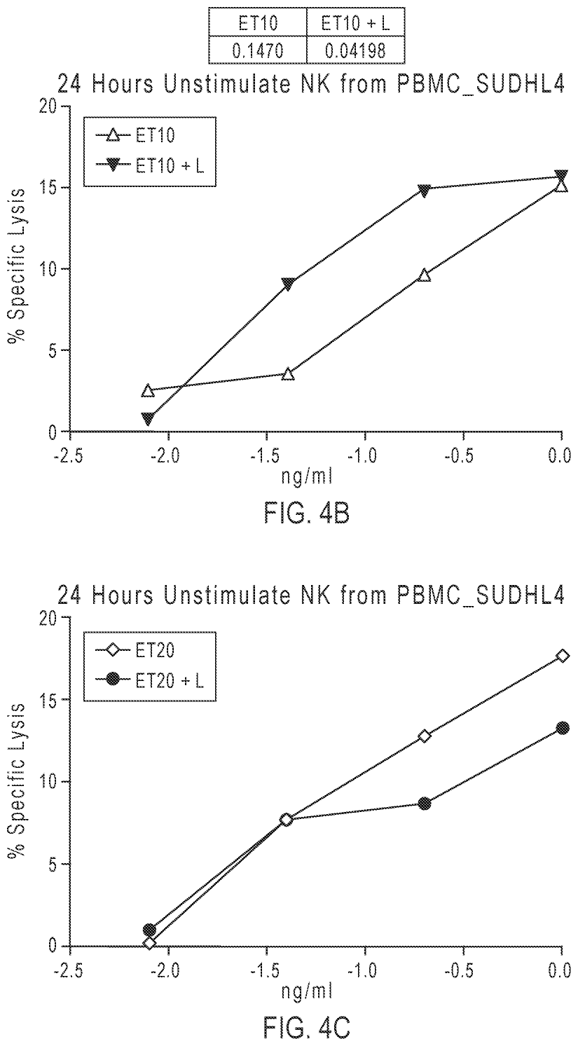

FIGS. 4A-4C and 9B and 9D show results where isolated pNK cells were included as effector cells. Pretreatment of PBMC with lenalidomide for 24 hrs, followed by pNK isolation, resulted in enhanced VAY736-induced ADCC, more so with 10:1 and 20:1 conditions (FIG. 4A-4C), and 1:1 and 1:3 with 72 hr Len pretreatment (FIG. 9B).

### 8.2.3. Discussion

The in vitro ADCC assay used in this Example involved various effector cells, and enhanced VAY736-induced ADCC was seen with all effector cell types at various culture conditions. Interestingly, it was found that CD3+ cells were needed for this enhancement of ADCC by lenalidomide. Without being bound by theory, it is believed that this observation may be due to the positive effect lenalidomide has on T cell IL2 secretion; IL2 would in turn provide positive activation signals for NK cells. In conclusion, these in vitro studies support the combination of VAY736 and lenalidomide for treating B-cell malignancies, such as DLBCL.

### 8.3. Example 3: Phase Ib Open-Label Study of VAY736 and Ibrutinib in Patients with Chronic Lymphocytic Leukemia (CLL)

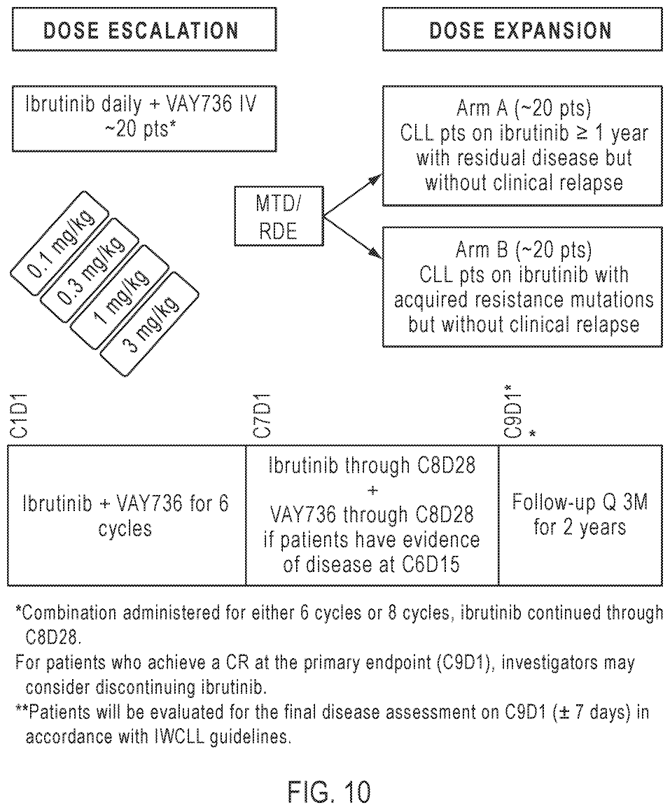

A clinical study (CVAY736Y2102) is ongoing to determine the safe and tolerable dose of VAY736 for use in combination with ibrutinib for treating chronic lymphocytic leukemia (CLL) and to explore preliminary efficacy of the combination. The treatment regimens for the study are schematically shown in FIG. 10, and include VAY736 dose levels of 0.3 mg/kg, 1 mg/kg, 3 mg/kg, and 9 mg/kg.

A total of 15 patients (median age: 65 years; ECOG PS 0: 93%) were treated by the data cutoff. Overall, 11 patients completed and 3 discontinued combination treatment (primarily due to disease progression); 1 patient remains on treatment. Most patients (73%) had an ibrutinib resistance mutation at baseline (mainly [82%] BTKC481) and 33% had received ≥4 prior regimens (median: 3, range: 1-5); median duration of prior ibrutinib was 4.1 y (range: 0.2-8.3). Baseline cytogenetics were (not mutually exclusive): 27% del(17)(p13.1), 80% unmutated IGHV, 80% stimulated complex karyotypes (≥3 abnormalities), 60% del(13)(q14), and 7%+12.

No dose-limiting toxicities have been observed and the MTD has not been reached. A total of 14 (93%) patients experienced an AE regardless of cause. Four (27%) patients experienced AEs of grade≥3, including decreased neutrophil count (n=3), hypophosphatemia (n=2), decreased white blood cell count, leukocytosis, increased lymphocyte count, hypertension, hypokalemia, and hypomagnesemia (n=1 each).

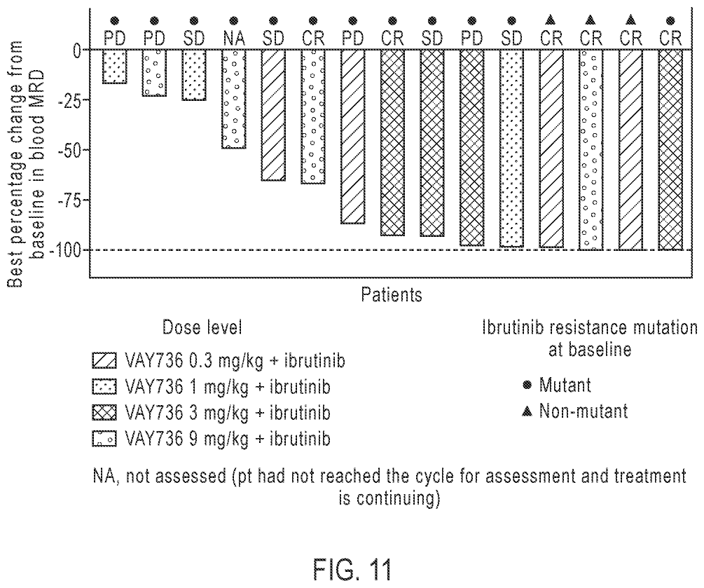

The overall response at cycle 9, date 1 (C9D1) was CR in 6 (40%) patients, SD in 4 (27%) patients, PD in 4 (27%) patients, and not assessed in 1 (7%) patient (still on treatment). The mean baseline CLL cells in bone marrow for the CR, SD, and PD groups were 27% (range: 0.8-60.6%), 13% (range: 2.5-27%), and 66% (range: 47-77.9%). Three (20%) patients with CR achieved MRD-negativity and were able to discontinue CLL-directed therapy including ibrutinib; they remained in CR for 1-16 months after ibrutinib discontinuation. The median percentage change from baseline in blood MRD was −92.8% (range: −100%; −16.7%; FIG. 11) and in bone marrow MRD was −89.6% (range: −100%; −32.6%). Of the patients who had baseline ibrutinib-resistance mutations and C9D1 assessments, 1 patient (1/6) tested negative for ibrutinib resistance mutations at C9D1. None of the patients who were ibrutinib-resistance mutation negative at baseline (4/4) developed mutations by C9D1.

VAY736 concentration increased with dose, accumulated after repeated dosing in combination with ibrutinib, and achieved linear PK at 3 mg/kg or above. Tissue receptor occupancy was >99% for VAY736 doses of 3 mg/kg or above. Free BAFF was accumulated to steady state with no dose relationship.

VAY736+ibrutinib had an acceptable safety profile and demonstrated promising preliminary activity in patients with R/R CLL on ibrutinib, providing clinical evidence of a potential to discontinue ibrutinib by VAY736 add-on therapy. Further investigation of this combination including in patients on first line ibrutinib and other ibrutinib combinations is ongoing.

### 8.4. Example 4: Phase Ib Open-Label Study of VAY736 and Lenalidomide in Patients with B-Cell Non-Hodgkin Lymphoma (NHL)

The preclinical data for VAY736 (see, e.g., Examples 1 and 2) along with encouraging ORR and PFS in patients with heavily pre-treated CLL (see, e.g., Example 3) support studies of VAY736 in the NHL population, as a single agent, or as a “backbone” agent in combination with additional agents. This Example evaluates the safety and tolerability of VAY736 and VAY736 in combination with lenalidomide (an IMiD having direct and indirect effects on NHL cells and single-agent activity in relapsed/refractory B-cell NHL), as well as the anti-tumor activity of VAY736 as a single agent and in combination with lenalidomide.

### 8.4.1. Materials and Methods

This is a phase Ib, multi-center, open-label study with multiple treatment arms. The study is comprised of a dose escalation part and a dose expansion part.

In this study, VAY736 is considered as “backbone”. The combination of a backbone and lenalidomide as partner therapy constitutes a treatment arm. VAY736 is used as the initial backbone single agent to which the lenalidomide partner is added to comprise a doublet treatment arm. During the dose escalation part of each treatment arm, patients are treated with VAY736 alone or in combination with partner investigational drug lenalidomide.

Each treatment arm enrolls cohorts of three to six patients with NHL (or specified NHL subtype, such as DLBCL) treated until the MTD is reached or a lower RD is established.

The platform study design methodology has been successfully applied as an accelerated mechanism to evaluate multiple compounds in various disease, and has been successfully implemented in recent clinical studies (Ventz et al. 2017, Saville et al. 2016, Renfro et al. 2017, Berry et al. 2015, Woodcock et al. 2017).

Dose escalation and determination of the MTD/RD is guided by a Bayesian Hierarchical Logistic Regression Model (BHLRM) with Escalation with Overdose Control (EWOC) criteria.

Each dose expansion arm will enroll approximately 20 patients. The expansion part for a given combination treatment may enrolled additional patients depending upon anti-tumor activity.

Multiple combination treatment arms are explored over time, for example:
Arm 1A: VAY736 single agent dose escalation,
Arm 1B: VAY736 single agent dose expansion,
Arm 2A: VAY736+lenalidomide dose escalation,
Arm 2B: VAY736+lenalidomide dose expansion, etc.

### 8.4.1.1. Dosing Regimen

The dosing cycle is 28 days. In VAY736 single agent dose escalation, patients receive VAY736 intravenously (i.v.) once every 2 weeks (Day 1 and Day 15 of each cycle) (Q2W). In the VAY736+lenalidomide dose escalation, lenalidomide is 25 mg QD PO, given on Days 1-21 of 28-day cycles for a maximum of 12 cycles; patients then continue on VAY736 monotherapy. Alternative dosing schedules (e.g. once very week (QW) or once every four weeks (Q4W)) may also be evaluated if supported by emerging clinical data. During the dose expansion part of the study, lenalidomide is continued at the same dose schedule as determined in the dose escalation part.

Dose of lenalidomide during dose expansion is 25 mg or highest tolerated dose.

Doses of VAY736 during dose escalation are 1 mg/kg i.v., 3 mg/kg i.v., 6 mg/kg i.v., 12 mg/kg i.v. and 20 mg/kg i.v.

In general, concomitant medications and therapies deemed necessary for the supportive care (e.g. such as anti-emetics, anti-diarrhea) and safety of the patient are allowed. Limited-field palliative radiotherapy to a non-target lesion is allowed.

8.4.1.2. Inclusion criteria applicable for all treatment arms
1. Histologically confirmed diagnosis of B-non-Hodgkin lymphoma (NHL), including all subtypes of Diffuse large B-cell lymphoma (DLBCL), follicular lymphoma (FL), marginal zone lymphoma (MZL), and mantle cell lymphoma (MCL) per WHO 2016 criteria (Swerdlow et al 2016).
2. Patients with NHL must have received and failed standard of care therapy (at least one prior line of systemic therapy, including an anti-CD20 therapy for NHL, but no more than 5 prior lines of therapy), or be intolerant or ineligible to other approved therapies including autologous stem cell transplantation (ASCT). Patients with indolent lymphoma must have received and failed standard of care therapy or be intolerant or ineligible to approved therapies and must be in need of therapeutic intervention.
3. Patients who are refractory to or have relapsed after prior CAR T-cell therapy will be permitted on this trial 30 days after CART infusion and meet protocol defined Inclusion/Exclusion criteria. Treatment will be permitted after confirmation of disease progression post CAR T-cell therapy.
4. Eastern Cooperative Oncology Group (ECOG) performance status ≤2
5. Measurable disease at time of enrollment:
   a. Nodal lesion(s)≥15 mm in the long axis, regardless of the length of the short axis and/or
   b. Extranodal lesions≥10 mm in long and short axis

### 8.4.1.3. Exclusion Criteria Applicable for all Treatment Arms

Patients meeting any of the following criteria are not eligible for inclusion in this study:
1. Patients with out of range laboratory values defined as:
   Serum creatinine>1.5× upper limit of normal (ULN) and/or calculated creatinine clearance<45 mL/min for VAY736 single agent arms using Cockcroft-Gault formula (for lenalidomide arms, see Section 8.4.1.4).
   Aspartate transaminase (AST)>3.0×ULN, except for patients with liver involvement, who are excluded if (AST)>5.0×ULN
   Alanine transaminase (ALT)>3.0×ULN, except for patients with liver involvement, who are excluded if (ALT)>5.0×ULN
   Total bilirubin>1.5×ULN, except for patients with Gilbert's syndrome who are excluded if total bilirubin>3.0×ULN or direct bilirubin>1.5×ULN)
   Hemoglobin (Hgb)<8 g/dL (transfusion support may not be used within 7 days prior to the first dose of study treatment)
   Absolute neutrophil count (ANC)<1.0×10⁹/L independent of growth factor support within 7 days prior to first dose of study treatment
   Platelets<75×10⁹/L without transfusion support within 7 days of the first dose of study drug. Patients with transfusion dependent thrombocytopenia are excluded.
2. Presence or history of central nervous system involvement by lymphoma.
3. Patients who have received anti-cancer therapies within the following time frames prior to the first dose of study treatment
   Conventional cytotoxic chemotherapy: ≤5 half-lives or ≤4 weeks (whichever is longer)
   Biologic therapy (e.g. antibodies): ≤4 weeks
   Non-cytotoxic small molecule therapeutics: ≤5 half-lives or ≤2 weeks (whichever is longer)
   Radiation therapy (palliative setting is allowed): ≤4 weeks: Palliative radiotherapy to a limited field, such as for the treatment of bone pain or focally painful tumor mass is allowed. To allow for assessment of response to treatment, patients must have remained measurable disease that has not been irradiated
   Major surgery: 52 weeks (mediastinoscopy, insertion of a central venous access device and insertion of a feeding tube are not considered major surgery)
   Autologous stem cell transplant within 12 weeks before first dose of study drug
4. Patients who have undergone prior allogeneic stem cell transplant before the first dose of study drug
5. Investigational medicinal product within the last 30 days or five half-lives or until the expected pharmacodynamic effect has returned to baseline (e.g. biologics), prior to screening, whichever is longer or longer if required by local regulations
6. Ongoing immunosuppressive therapy, including systemic corticosteroids for treatment of lymphoma. Subjects may use topical or inhaled corticosteroids as therapy for comorbid conditions and low-dose systemic corticosteroids (≤25 mg/day of prednisone or equivalent) for endocrine or rheumatologic conditions. During study participation, subjects may receive systemic or other corticosteroids as pretreatment for VAY736 infusions or as needed for treatment-emergent comorbid conditions.
7. Receipt of attenuated vaccine within a 30 day period before VAY736 treatment
8. History of hypersensitivity to VAY736 or any of its excipients or to drugs of similar chemical classes (e.g. mAb)
9. Impaired cardiac function or clinically significant cardiac disease, including any of the following:
   Clinically significant and/or uncontrolled heart disease such as congestive heart failure requiring treatment (NYHA Grade≥2), uncontrolled hypertension or clinically significant arrhythmia.
   QTcF>480 msec on screening ECG or congenital long QT syndrome
   Acute myocardial infarction or unstable angina pectoris<3 months prior to study entry.
10. Subjects with history of drug-induced pneumonitis or current pneumonitis.
11. Patients with impaired hepatic function as defined by Childs-Pugh class B or C.
12. History of stroke or intracranial hemorrhage within 6 months prior to start of study drug
13. Evidence of active ongoing systemic bacterial, mycobacterial, fungal, or viral infection at the time of study enrollment. Subjects with localized fungal infections of skin or nails are eligible. Subjects may be receiving prophylactic antiviral or antibacterial therapies at the discretion of the investigator.
14. Malignant disease other than that being treated in the study. Exceptions include basal cell carcinoma of the skin or squamous cell carcinoma of the skin that has undergone potentially curative therapy or in situ cervical cancer or other tumors that will not affect life expectancy.
15. All acute toxic effects of any prior antitumor therapy (including lenalidomide) resolved to ≤CTCAE v5.0 G1 before study enrollment (with the exception of alopecia, G2 neurotoxicity, or G2 or G3 bone marrow parameters)
16. Patient has known history of HIV infection or tested positive for HIV infection (patient is positive in either HIV1/2 immunoassay and/or HIV RNA viral load)
17. Active hepatitis C infection defined by a positive RNA PCR test and/or hepatitis B infection defined as:
   Positive serology for hepatitis B surface antigen (HbsAg)
   Positive serology for hepatitis B core antibody (HbcAb), except if all 3 following criteria are met:
      i. HBV DNA negative
      ii. Prophylactic treatment (with nucleos/tide) initiated latest on Day 1 and continued until 12 months after last treatment
      iii. Hepatitis B monitoring is implemented: HbsAg (and HBV DNA) tested every 4 weeks until the end of prophylactic treatment.
   Patients whose disease is controlled under antiviral therapy should not be excluded. Patients who are solely HBsAb positive are eligible.
18. Patients who required discontinuation of treatment due to treatment-related toxicities during prior therapy directed against the same target as the drug(s) under study in this protocol.
19. Suspected or confirmed COVID-19 infection unless vaccinated or serologically positive or adequately treated more than 30 days before signing study informed consent. COVID-19 testing is mandatory.
20. Severe and/or uncontrolled medical conditions that in the investigator's opinion could affect the safety of the individual or impair the assessment of study result
21. Pregnant or nursing (lactating) women
22. Women of child-bearing potential, defined as all women physiologically capable of becoming pregnant, unless they are using highly effective methods of contraception (VAY736 only arms) or two methods of reliable contraception (a highly effective method of contraception and 1 additional effective contraceptive method (male latex or synthetic condom, diaphragm, or cervical cap) if taking lenalidomide) while taking study treatment and for 4 months after stopping medication. For lenalidomide arm, contraception must begin 4 weeks prior to Cycle 1 Day 1. This duration may be increased based on emerging PK data. Highly effective contraception methods include:
   Total abstinence (when this is in line with the preferred and usual lifestyle of the subject. Periodic abstinence (e.g. calendar, ovulation, symptothermal, post-ovulation methods) and withdrawal are not acceptable methods of contraception.
   Female sterilization (have had surgical bilateral oophorectomy with or without hysterectomy), total hysterectomy, or tubal ligation at least six weeks before taking study treatment. In case of oophorectomy alone, only when the reproductive status of the woman has been confirmed by follow-up hormone level assessment.
   Male sterilization (at least 6 months prior to screening). For female subjects on the study, the vasectomized male partner should be the sole partner for that subject.
   Use of oral (estrogen and progesterone), injected, or implanted hormonal methods of contraception or placement of an intrauterine device (IUD) or intrauterine system (IUS), or other forms of hormonal contraception that have comparable efficacy (failure rate<1%), for example, hormone vaginal ring or transdermal hormone contraception.
      In case of use of oral contraception, women should have been stable on the same pill for a minimum of 3 months before taking study treatment.
      Women are considered post-menopausal and not of child bearing potential if they have had 12 months of natural (spontaneous) amenorrhea with an appropriate clinical profile (e.g. age-appropriate (generally age from 40 to 59 years), history of vasomotor symptoms, [e.g. hot flushes] in the absence of other medical justification) or have had surgical bilateral oophorectomy (with or without hysterectomy), total hysterectomy or bilateral tubal ligation at least six weeks ago. In the case of oophorectomy alone, only when the reproductive status of the woman has been confirmed by follow-up hormone level assessment is she considered not of child-bearing potential.
23. Sexually active males unwilling to use a latex or synthetic condom during intercourse while taking study treatment and for 150 days (five predicted half-lives of VAY736 plus 90 days) after stopping study treatment, even if they have undergone a successful vasectomy. This duration may be increased based on emerging PK data. A condom is required for all sexually active male patients to prevent them from fathering a child AND to prevent delivery of study treatment via seminal fluid to their partner. In addition, male patients must not donate sperm for the time period specified above.

### 8.4.1.4. Exclusion Criteria Specific to Treatment Arms Containing Lenalidomide Arms

Patients meeting any of the following criteria are not eligible for inclusion in the lenalidomide treatment arms:
101. Ongoing or prior treatment with lenalidomide or thalidomide or thalidomide analogues.
102. Primary refractory DLBCL, defined as a response of less than a PR (partial response) to or progression during or within 6 months of frontline therapy or first line therapy
103. History of double-hit or triple hit DLBCL if already known (simultaneous detection of MYC with BCL2 or BCL6 or both translations)
104. Known allergy to lenalidomide or to compounds with similar chemical structure of lenalidomide or derivatives (e.g. thalidomide, pomalidomide) and/or their excipients
105. Prior history of angioedema or severe cutaneous reactions including Steven-Johnson syndrome (SJS), toxic epidermal necrolysis (TEN), and drug reaction with eosinophilia and systemic symptoms (DRESS) or a G4 rash associated with lenalidomide or thalidomide treatment
106. Prior history of tumor lysis syndrome with lenalidomide or thalidomide treatment
107. Serum creatinine>1.5× upper limit of normal (ULN) and/or calculated creatinine clearance<61 mL/min for lenalidomide arms using Cockcroft-Gault formula (or as per country package insert for lenalidomide 25 mg QD dose but not lower than <61 mL/min)
108. Patients who are at risk for a thromboembolic event and are not willing to take prophylaxis for it.
109. Impairment of gastrointestinal (GI) function or GI disease that may significantly alter the absorption of oral lenalidomide (e.g., ulcerative diseases, uncontrolled nausea, vomiting, diarrhea, malabsorption syndrome, small bowel resection or presence of more than or equal to Grade 2 GI-related toxicity due to prior therapy).
110. Unable or unwilling to agree to not donate blood during treatment with lenalidomide and for 4 weeks following discontinuation of lenalidomide (due to risk that the blood may be given to a pregnant patient whose fetus must not be exposed to lenalidomide)
111. Unable or unwilling to swallow the oral drug as per dosing schedule.

### 8.4.1.5. Objectives and Endpoints

The objectives and endpoints for all arms are shown in the table below.

| Objective(s) | Endpoint(s) |
|---|---|
| Primary Objective(s) | Endpoint(s) for primary objective(s) |
| 1. The primary objective of this study is to | Safety: |
| evaluate the safety and tolerability in patients with | Incidence of DLTs (dose escalation only) |
| Non Hodgkin lymphoma and identify a maximum | Incidence and severity of AEs and serious |
| tolerated dose (MTD) and/or recommended dose | adverse events (SAEs), changes in laboratory |
| (RD) of VAY736 single agent and in combination | values, vital signs and ECGs graded as per |
| | NCI CTCAE v5.0 |
| | Tolerability |
| | Dose interruptions, reductions and dose |
| | intensity |
| Secondary Objective(s) | Endpoint(s) for secondary objective(s) |
| To estimate anti-tumor activity of VAY736 single | Overall response rate (ORR), Best overall |
| agent and combination regimens | response (BOR) rate, Progression free survival |
| | (PFS), Duration of response (DOR), and Time to |
| | progression (TTP) by Lugano Classification |
| | (FDG-PET CT Scan) |
| To assess pharmacokinetics of VAY736 single | |
| agent and combination partners | |
| To assess immunogenicity of VAY736 and in combination | Serum concentrations of VAY736 and plasma |
| | concentrations of each combination partner and |
| | derived PK parameters |
| | Antidrug antibodies (ADA) prevalence at baseline |
| | and post-baseline timepoints |

### 8.5. Example 5: Depletion of Normal B Cells by Ianalumab in Mouse

The effect of ianalumab on healthy B-cell levels in mice was evaluated in a repeat dose toxicity study.

### 8.5.1. Materials and Methods

CD-1 mice were administered 0 mg/kg or 100 mg/kg of ianalumab by intravenous administration weekly for 13 weeks, followed by an 11 week recovery period.

### 8.5.2. Results

In CD-1 mice administered 100 mg/kg of ianalumab, 70-90% of mature B cell depletion was observed. B cell levels recovered during the recovery period.

### 8.6. Example 6: Depletion of Normal B Cells by Ianalumab in Cynomolgus Monkey

A rising single i.v. dose range finding (DRF), toxicity and TK/PD study and three repeated dose toxicity studies were performed with ianalumab in cynomolgus monkeys. B-cell levels were evaluated in the studies.

In the single dose study, ianalumab at doses of 0.4 mg/kg and higher induced depletion of B cells. Ianalumab was well tolerated.

Across three repeated dose studies, B cell depletion was observed at all dose levels.

Taken together, the mouse and cynomolgus monkey studies show that ianalumab depletes healthy B-cells in vivo. A similar effect of ianalumab on healthy B-cell cells in human is expected.

### 9. SPECIFIC EMBODIMENTS, CITATION OF REFERENCES

While various specific embodiments have been illustrated and described, it will be appreciated that various changes can be made without departing from the spirit and scope of the disclosure(s). The present disclosure is exemplified by the numbered embodiments set forth below.

1. An anti-BAFFR antibody or a binding fragment thereof for use in the treatment of B cell malignancy in a subject in need thereof, wherein the anti-BAFFR antibody or binding fragment thereof is to be administered at a therapeutically effective dose.

2. The anti-BAFFR antibody or a binding fragment thereof for use according to embodiment 1, wherein the anti-BAFFR antibody or binding fragment thereof comprises CDR-H1, CDR-H2, and CDR-H3 having the amino acid sequences of SEQ ID NO:3, SEQ ID NO:4, and SEQ ID NO:5, and CDR-L1, CDR-L2, and CDR-L3 having the amino acid sequences of SEQ ID NO:6, SEQ ID NO:7, and SEQ ID NO:8, respectively.

3. The anti-BAFFR antibody or a binding fragment thereof for use according to embodiment 1 or embodiment 2, wherein the anti-BAFFR antibody or binding fragment thereof comprises a heavy chain variable region having the amino acid sequence of SEQ ID NO: 1 and a light chain variable region having the amino acid sequence of SEQ ID NO: 2.

4. The anti-BAFFR antibody or a binding fragment thereof for use according to any one of embodiments 1 to 3, wherein the anti-BAFFR antibody or binding fragment thereof is ianalumab or a binding fragment thereof.

5. The anti-BAFFR antibody or a binding fragment thereof for use according to any one of embodiments 1 to 4, wherein the anti-BAFFR antibody or binding fragment thereof is to be administered at a dose of 0.1 mg/kg to 20 mg/kg.

6. The anti-BAFFR antibody or a binding fragment thereof for use according to embodiment 5, wherein the anti-BAFFR antibody or binding fragment thereof is to be administered at a dose of 1 mg/kg to 10 mg/kg.

7. The anti-BAFFR antibody or a binding fragment thereof for use according to embodiment 5, wherein the anti-BAFFR antibody or binding fragment thereof is to be administered at a dose of 5 mg/kg to 15 mg/kg.

8. The anti-BAFFR antibody or a binding fragment thereof for use according to embodiment 5, wherein the anti-BAFFR antibody or binding fragment thereof is to be administered at a dose of 10 mg/kg to 20 mg/kg.

9. The anti-BAFFR antibody or a binding fragment thereof for use according to embodiment 5, wherein the anti-BAFFR antibody or binding fragment thereof is to be administered at a dose of 1 mg/kg.

10. The anti-BAFFR antibody or a binding fragment thereof for use according to embodiment 5, wherein the anti-BAFFR antibody or binding fragment thereof is to be administered at a dose of 3 mg/kg.

11. The anti-BAFFR antibody or a binding fragment thereof for use according to embodiment 5, wherein the anti-BAFFR antibody or binding fragment thereof is to be administered at a dose of 6 mg/kg.

12. The anti-BAFFR antibody or a binding fragment thereof for use according to embodiment 5, wherein the anti-BAFFR antibody or binding fragment thereof is to be administered at a dose of 9 mg/kg.

13. The anti-BAFFR antibody or a binding fragment thereof for use according to embodiment 5, wherein the anti-BAFFR antibody or binding fragment thereof is to be administered at a dose of 12 mg/kg.

14. The anti-BAFFR antibody or a binding fragment thereof for use according to embodiment 5, wherein the anti-BAFFR antibody or binding fragment thereof is to be administered at a dose of 20 mg/kg.

15. The anti-BAFFR antibody or a binding fragment thereof for use according to any one of embodiments 1 to 14, wherein the anti-BAFFR antibody or binding fragment thereof is to be administered to a subject in need thereof once every two weeks (+/−3 days).

16. The anti-BAFFR antibody or a binding fragment thereof for use according to embodiment 15, wherein the anti-BAFFR antibody or binding fragment thereof is to be administered on day 1+/−3 days and day 15+/−3 days of a 28-day dosing cycle.

17. The anti-BAFFR antibody or a binding fragment thereof for use according to embodiment 15, wherein the anti-BAFFR antibody or binding fragment thereof is to be administered on day 1+/−2 days and day 15+/−2 days of a 28-day dosing cycle.

18. The anti-BAFFR antibody or a binding fragment thereof for use according to embodiment 15, wherein the anti-BAFFR antibody or binding fragment thereof is to be administered on day 1+/−1 day and day 15+/−1 day of a 28-day dosing cycle.

19. The anti-BAFFR antibody or a binding fragment thereof for use according to embodiment 15, wherein the anti-BAFFR antibody or binding fragment thereof is to be administered on day 1 and day 15 of a 28-day dosing cycle.

20. The anti-BAFFR antibody or a binding fragment thereof for use according to any one of embodiments 1 to 14, wherein the anti-BAFFR antibody or binding fragment thereof is to be administered to a subject in need thereof once every week (+/−3 days).

21. The anti-BAFFR antibody or a binding fragment thereof for use according to embodiment 20, wherein the anti-BAFFR antibody or binding fragment thereof is to be administered on day 1+/−3 days, day 8+/−3 days, day 15+/−3 days, and day 22+/−3 days of a 28-day dosing cycle.

22. The anti-BAFFR antibody or a binding fragment thereof for use according to embodiment 20, wherein the anti-BAFFR antibody or binding fragment thereof is to be administered on day 1+/−2 days, day 8+/−2 days, day 15+/−2 days, and day 22+/−2 days of a 28-day dosing cycle.

23. The anti-BAFFR antibody or a binding fragment thereof for use according to embodiment 20, wherein the anti-BAFFR antibody or binding fragment thereof is to be administered on day 1+/−1 day, day 8+/−1 day, day 15+/−1 day, and day 22+/−1 day of a 28-day dosing cycle.

24. The anti-BAFFR antibody or a binding fragment thereof for use according to embodiment 20, wherein the anti-BAFFR antibody or binding fragment thereof is to be administered on day 1, day 8, day 15, and day 22 of a 28-day dosing cycle.

25. The anti-BAFFR antibody or a binding fragment thereof for use according to any one of embodiments 1 to 14, wherein the anti-BAFFR antibody or binding fragment thereof is to be administered to a subject in need thereof once every 4 weeks (+/−3 days).

26. The anti-BAFFR antibody or a binding fragment thereof for use according to embodiment 25, wherein the anti-BAFFR antibody or binding fragment thereof is to be administered on day 1+/−3 days of a 28-day dosing cycle.

27. The anti-BAFFR antibody or a binding fragment thereof for use according to embodiment 26, wherein the anti-BAFFR antibody or binding fragment thereof is to be administered on day 1+/−2 days of a 28-day dosing cycle.

28. The anti-BAFFR antibody or a binding fragment thereof for use according to embodiment 26, wherein the anti-BAFFR antibody or binding fragment thereof is to be administered on day 1+/−1 day of a 28-day dosing cycle.

29. The anti-BAFFR antibody or a binding fragment thereof for use according to embodiment 20, wherein the anti-BAFFR antibody or binding fragment thereof is to be administered on day 1 of a 28-day dosing cycle.

30. The anti-BAFFR antibody or a binding fragment thereof for use according to any one of embodiments 15 to 29 wherein the anti-BAFFR antibody or a binding fragment thereof is to be administered for 12 or more 28-day cycles.

31. The anti-BAFFR antibody or a binding fragment thereof for use according to any one of embodiments 1 to 30, wherein the anti-BAFFR antibody or binding fragment thereof is to be administered intravenously to a subject in need thereof.

32. The anti-BAFFR antibody or a binding fragment thereof for use according to any one of embodiments 1 to 31, wherein the anti-BAFFR antibody or binding fragment thereof is to be administered as monotherapy for the B cell malignancy.

33. The anti-BAFFR antibody or a binding fragment thereof for use according to any one of embodiments 1 to 31, wherein the anti-BAFFR antibody or binding fragment thereof is to be administered in combination with one or more additional agents.

34. The anti-BAFFR antibody or a binding fragment thereof for use according to embodiment 33, wherein the one or more additional agents comprise an immunomodulatory imide drug (IMiD).

35. The anti-BAFFR antibody or a binding fragment thereof for use according to embodiment 34, wherein the IMiD is lenalidomide or a pharmaceutically acceptable salt thereof, thalidomide or a pharmaceutically acceptable salt thereof, pomalidomide or a pharmaceutically acceptable salt thereof, or iberdomide or a pharmaceutically acceptable salt thereof.

36. The anti-BAFFR antibody or a binding fragment thereof for use according to embodiment 34, wherein the IMiD is lenalidomide or a pharmaceutically acceptable salt thereof.

37. The anti-BAFFR antibody or a binding fragment thereof for use according to embodiment 36, wherein the lenalidomide or a pharmaceutically acceptable salt thereof is to be administered at a dose of 2.5 mg to 25 mg.

38. The anti-BAFFR antibody or a binding fragment thereof for use according to embodiment 37, wherein the lenalidomide or a pharmaceutically acceptable salt thereof is to be administered at a dose of 2.5 mg.

39. The anti-BAFFR antibody or a binding fragment thereof for use according to embodiment 37, wherein the lenalidomide or a pharmaceutically acceptable salt thereof is to be administered at a dose of 5 mg.

40. The anti-BAFFR antibody or a binding fragment thereof for use according to embodiment 37, wherein the lenalidomide or a pharmaceutically acceptable salt thereof is to be administered at a dose of 15 mg.

41. The anti-BAFFR antibody or a binding fragment thereof for use according to embodiment 37, wherein the lenalidomide or a pharmaceutically acceptable salt thereof is to be administered at a dose of 20 mg.

42. The anti-BAFFR antibody or a binding fragment thereof for use according to embodiment 37, wherein the lenalidomide or a pharmaceutically acceptable salt thereof is to be administered at a dose of 25 mg.

43. The anti-BAFFR antibody or a binding fragment thereof for use according to any one of embodiments 36 to 42, wherein the lenalidomide or a pharmaceutically acceptable salt thereof is to be administered to a subject in need thereof once a day.

44. The anti-BAFFR antibody or a binding fragment thereof for use according to any one of embodiments 36 to 43, wherein the lenalidomide or a pharmaceutically acceptable salt thereof is to be administered orally to a subject in need thereof.

45. The anti-BAFFR antibody or a binding fragment thereof for use according to any one of embodiments 36 to 44, wherein the lenalidomide or a pharmaceutically acceptable salt thereof is to be administered on days 1 to 21 of a 28-day cycle.

46. The anti-BAFFR antibody or a binding fragment thereof for use according to embodiment 45, wherein the lenalidomide or a pharmaceutically acceptable salt thereof is to be administered for up to 12 cycles.

47. The anti-BAFFR antibody or a binding fragment thereof for use according to embodiment 46, which is to be administered as monotherapy following the last administration of lenalidomide or pharmaceutically acceptable salt.

48. The anti-BAFFR antibody or a binding fragment thereof for use according to any one of embodiments 33 to 47, wherein the one or more additional agents comprise an anti-emetic agent.

49. The anti-BAFFR antibody or a binding fragment thereof for use according to any one of embodiments 33 to 48, wherein the one or more additional agents comprise an anti-diarrheal agent.

50. A method of treating a subject having a B cell malignancy, comprising administering therapeutically effective dose of an anti-BAFFR antibody or a binding fragment thereof to the subject.

51. The method of embodiment 50, wherein the anti-BAFFR antibody or binding fragment thereof comprises CDR-H1, CDR-H2, and CDR-H3 having the amino acid sequences of SEQ ID NO:3, SEQ ID NO:4, and SEQ ID NO:5, and CDR-L1, CDR-L2, and CDR-L3 having the amino acid sequences of SEQ ID NO:6, SEQ ID NO:7, and SEQ ID NO:8, respectively.

52. The method of embodiment 50 or embodiment 51, wherein the anti-BAFFR antibody or binding fragment thereof comprises a heavy chain variable region having the amino acid sequence of SEQ ID NO: 1 and a light chain variable region having the amino acid sequence of SEQ ID NO: 2.

53. The method of any one of embodiments 50 to 52, wherein the anti-BAFFR antibody or binding fragment thereof is ianalumab or a binding fragment thereof.

54. The method of any one of embodiments 50 to 53, wherein the anti-BAFFR antibody or binding fragment thereof is administered at a dose of 0.1 mg/kg to 20 mg/kg.

55. The method of embodiment 54, wherein the anti-BAFFR antibody or binding fragment thereof is administered at a dose of 1 mg/kg to 10 mg/kg.

56. The method of embodiment 54, wherein the anti-BAFFR antibody or binding fragment thereof is administered, wherein the anti-BAFFR antibody or binding fragment thereof is administered at a dose of 5 mg/kg to 15 mg/kg.

57. The method of embodiment 54, wherein the anti-BAFFR antibody or binding fragment thereof is administered, wherein the anti-BAFFR antibody or binding fragment thereof is administered at a dose of 10 mg/kg to 20 mg/kg.

58. The method of embodiment 54, wherein the anti-BAFFR antibody or binding fragment thereof is administered, wherein the anti-BAFFR antibody or binding fragment thereof is administered at a dose of 1 mg/kg.

59. The method of embodiment 54, wherein the anti-BAFFR antibody or binding fragment thereof is administered, wherein the anti-BAFFR antibody or binding fragment thereof is administered at a dose of 3 mg/kg.

60. The method of embodiment 54, wherein the anti-BAFFR antibody or binding fragment thereof is administered, wherein the anti-BAFFR antibody or binding fragment thereof is administered at a dose of 6 mg/kg.

61. The method of embodiment 54, wherein the anti-BAFFR antibody or binding fragment thereof is administered, wherein the anti-BAFFR antibody or binding fragment thereof is administered at a dose of 9 mg/kg.

62. The method of embodiment 54, wherein the anti-BAFFR antibody or binding fragment thereof is administered, wherein the anti-BAFFR antibody or binding fragment thereof is administered at a dose of 12 mg/kg.

63. The method of embodiment 54, wherein the anti-BAFFR antibody or binding fragment thereof is administered, wherein the anti-BAFFR antibody or binding fragment thereof is administered at a dose of 20 mg/kg.

64. The method of any one of embodiments 50 to 63, wherein the anti-BAFFR antibody or binding fragment thereof is administered to the subject once every two weeks (+/−3 days).

65. The method of embodiment 64, wherein the anti-BAFFR antibody or binding fragment thereof is administered on day 1+/−3 days and day 15+/−3 days of a 28-day dosing cycle.

66. The method of embodiment 64, wherein the anti-BAFFR antibody or binding fragment thereof is administered on day 1+/−2 days and day 15+/−2 days of a 28-day dosing cycle.

67. The method of embodiment 64, wherein the anti-BAFFR antibody or binding fragment thereof is administered on day 1+/−1 day and day 15+/−1 day of a 28-day dosing cycle.

68. The method of embodiment 64, wherein the anti-BAFFR antibody or binding fragment thereof is administered on day 1 and day 15 of a 28-day dosing cycle.

69. The method of any one of embodiments 50 to 63, wherein the anti-BAFFR antibody or binding fragment thereof is administered to the subject once every week (+/−3 days).

70. The method of embodiment 69, wherein the anti-BAFFR antibody or binding fragment thereof is administered on day 1+/−3 days, day 8+/−3 days, day 15+/−3 days, and day 22+/−3 days of a 28-day dosing cycle.

71. The method of embodiment 69, wherein the anti-BAFFR antibody or binding fragment thereof is administered on day 1+/−2 days, day 8+/−2 days, day 15+/−2 days, and day 22+/−2 days of a 28-day dosing cycle.

72. The method of embodiment 69, wherein the anti-BAFFR antibody or binding fragment thereof is administered on day 1+/−1 day, day 8+/−1 day, day 15+/−1 day, and day 22+/−1 day of a 28-day dosing cycle.

73. The method of embodiment 69, wherein the anti-BAFFR antibody or binding fragment thereof is administered on day 1, day 8, day 15, and day 22 of a 28-day dosing cycle.

74. The method of any one of embodiments 50 to 63, wherein the anti-BAFFR antibody or binding fragment thereof is administered to the subject once every 4 weeks (+/−3 days).

75. The method of embodiment 74, wherein the anti-BAFFR antibody or binding fragment thereof is administered on day 1+/−3 days of a 28-day dosing cycle.

76. The method of embodiment 74, wherein the anti-BAFFR antibody or binding fragment thereof is administered on day 1+/−2 days of a 28-day dosing cycle.

77. The method of embodiment 74, wherein the anti-BAFFR antibody or binding fragment thereof is administered on day 1+/−1 day of a 28-day dosing cycle.

78. The method of embodiment 74, wherein the anti-BAFFR antibody or binding fragment thereof is administered on day 1 of a 28-day dosing cycle.

79. The method of any one of embodiments 64 to 78, wherein the anti-BAFFR antibody or a binding fragment thereof is administered for 12 or more 28-day cycles.

80. The method of any one of embodiments 50 to 79, wherein the anti-BAFFR antibody or binding fragment thereof is administered intravenously to the subject.

81. The method of any one of embodiments 50 to 80, wherein the anti-BAFFR antibody or binding fragment thereof is administered as monotherapy for the B cell malignancy.

82. The method of any one of embodiments 50 to 80, wherein the anti-BAFFR antibody or binding fragment thereof is administered in combination with one or more additional agents.

83. The method of embodiment 82, wherein the one or more additional agents comprise an immunomodulatory imide drug (IMiD).

84. The method of embodiment 83, wherein the IMiD is lenalidomide or a pharmaceutically acceptable salt thereof, thalidomide or a pharmaceutically acceptable salt thereof, pomalidomide or a pharmaceutically acceptable salt thereof, or iberdomide or a pharmaceutically acceptable salt thereof.

85. The method of embodiment 84, wherein the IMiD is lenalidomide or a pharmaceutically acceptable salt thereof.

86. The method of embodiment 85, wherein the lenalidomide or a pharmaceutically acceptable salt thereof is administered to the subject at a dose of 2.5 mg to 25 mg.

87. The method of embodiment 86, wherein the lenalidomide or a pharmaceutically acceptable salt thereof is administered to the subject at a dose of 2.5 mg.

88. The method of embodiment 86, wherein the lenalidomide or a pharmaceutically acceptable salt thereof is administered to the subject at a dose of 5 mg.

89. The method of embodiment 86, wherein the lenalidomide or a pharmaceutically acceptable salt thereof is administered to the subject at a dose of 10 mg.

90. The method of embodiment 86, wherein the lenalidomide or a pharmaceutically acceptable salt thereof is administered to the subject at a dose of 15 mg.

91. The method of embodiment 86, wherein the lenalidomide or a pharmaceutically acceptable salt thereof is administered to the subject at a dose of 20 mg.

92. The method of embodiment 86, wherein the lenalidomide or a pharmaceutically acceptable salt thereof is administered to the subject at a dose of 25 mg.

93. The method of any one of embodiments 85 to 92, wherein the lenalidomide or a pharmaceutically acceptable salt thereof is administered to the subject once a day.

94. The method of any one of embodiments 85 to 93, wherein the lenalidomide or a pharmaceutically acceptable salt thereof is administered orally to the subject.

95. The method of any one of embodiments 85 to 94, wherein the lenalidomide or a pharmaceutically acceptable salt thereof is administered to the subject on days 1 to 21 of a 28-day cycle.

96. The method of embodiment 95, wherein the lenalidomide or a pharmaceutically acceptable salt thereof is administered for up to 12 cycles.

97. The method of embodiment 96, wherein the anti-BAFFR antibody or a binding fragment thereof is administered as monotherapy following the last administration of lenalidomide or pharmaceutically acceptable salt.

98. The method of any one of embodiments 82 to 97, wherein the one or more additional agents comprise an anti-emetic agent.

99. The method of any one of embodiments 82 to 98, wherein the one or more additional agents comprise an anti-diarrheal agent.

100. The method of any one of embodiments 82 to 99, which comprises administering the one or more additional agents to the subject.

101. A combination comprising (i) an anti-BAFFR antibody or a binding fragment thereof and (ii) one or more additional agents.

102. The combination of embodiment 101, wherein the anti-BAFFR antibody or binding fragment thereof comprises CDR-H1, CDR-H2, and CDR-H3 having the amino acid sequences of SEQ ID NO:3, SEQ ID NO:4, and SEQ ID NO:5, and CDR-L1, CDR-L2, and CDR-L3 having the amino acid sequences of SEQ ID NO:6, SEQ ID NO:7, and SEQ ID NO:8, respectively.

103. The combination of embodiment 101 or embodiment 102, wherein the anti-BAFFR antibody or binding fragment thereof comprises a heavy chain variable region having the amino acid sequence of SEQ ID NO: 1 and a light chain variable region having the amino acid sequence of SEQ ID NO: 2.

104. The combination of any one of embodiments 101 to 103, wherein the anti-BAFFR antibody or binding fragment thereof is ianalumab or a binding fragment thereof.

105. The combination of any one of embodiments 101 to 104, wherein the anti-BAFFR antibody or binding fragment thereof and the one or more additional agents are formulated in separate pharmaceutical compositions.

106. The combination of any one of embodiments 101 to 105, wherein the one or more additional agents comprise an immunomodulatory imide drug (IMiD).

107. The combination of embodiment 106, wherein the IMiD is lenalidomide or a pharmaceutically acceptable salt thereof, thalidomide or a pharmaceutically acceptable salt thereof, pomalidomide or a pharmaceutically acceptable salt thereof, or iberdomide or a pharmaceutically acceptable salt thereof.

108. The combination of embodiment 107, wherein the IMiD is lenalidomide or a pharmaceutically acceptable salt thereof.

109. The combination of any one of embodiments 101 to 108, wherein the one or more additional agents comprise an anti-emetic agent.

110. The combination of any one of embodiments 101 to 109, wherein the one or more additional agents comprise an anti-diarrheal agent.

111. The combination of any one of embodiments 101 to 110, for use in the treatment of B cell malignancy in a subject in need thereof.

112. Use of an anti-BAFFR antibody in the manufacture of a medicament for treating a subject having B cell malignancy, optionally wherein the medicament is for administration in combination with one or more additional agents, optionally wherein the one or more additional agents are one or more additional agents described in any one of embodiments 34 to 49.

113. Use an additional agent in the manufacture of a medicament for treating a subject having B cell malignancy, wherein the medicament is for administration in combination with an anti-BAFFR antibody or binding fragment thereof, optionally wherein the additional agent is an agent described in any one of embodiments 34 to 49.

114. The use of embodiment 112 or embodiment 113, wherein the anti-BAFFR antibody or binding fragment thereof is an anti-BAFFR antibody or binding fragment thereof described in any one of embodiments 2 to 4.

115. The use of any one of embodiments 112 to 114, wherein the anti-BAFFR antibody and/or one or more additional agents are formulated for administration according to the method of any one of embodiments 50 to 100.

116. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is a hematological cancer.

117. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is a malignant lymphoproliferative condition.

118. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is a plasma cell dyscrasia.

119. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is an acute leukemia.

120. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is B cell acute lymphocytic leukemia (B-ALL).

121. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is relapsed and/or refractory B cell acute lymphocytic leukemia (B-ALL).

122. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is a non-Hodgkin's lymphoma (NHL).

123. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is a relapsed and/or refractory non-Hodgkin's lymphoma (NHL).

124. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is chronic lymphocytic leukemia (CLL)/small lymphocytic lymphoma (SLL).

125. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is relapsed and/or refractory chronic lymphocytic leukemia (CLL)/small lymphocytic lymphoma (SLL).

126. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is follicular lymphoma (FL), optionally wherein the FL is small cell FL or large cell FL.

127. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is relapsed and/or refractory follicular lymphoma (FL), optionally wherein the FL is small cell FL or large cell FL.

128. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is mantle cell lymphoma (MCL).

129. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is relapsed and/or refractory mantle cell lymphoma (MCL).

130. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is diffuse large B-cell lymphoma (DLBCL).

131. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is relapsed and/or refractory diffuse large B-cell lymphoma (DLBCL).

132. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is Burkitt lymphoma.

133. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is lymphoplasmacytic lymphoma (Waldenstrom macroglobulinemia).

134. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is MALT lymphoma (mucosa-associated lymphoid tissue lymphoma).

135. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is marginal zone lymphoma (MZL).

136. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is extranodal marginal zone lymphoma (EMZL).

137. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is nodal marginal zone B-cell lymphoma (NZML).

138. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is splenic marginal zone B-cell lymphoma (SMZL).

139. The anti-BAFFR antibody or binding fragment, method, combination or use according to any one of embodiments 122 to 138, wherein the subject has failed at least one prior line of standard of care therapy.

140. The anti-BAFFR antibody or binding fragment, method, combination or use according to embodiment 139, wherein the subject has failed up to five prior lines of standard of care therapies.

141. The anti-BAFFR antibody or binding fragment, method, combination or use according to embodiment 139 or embodiment 140, wherein the subject has failed one prior line of standard of care therapy.

142. The anti-BAFFR antibody or binding fragment, method, combination or use according to embodiment 139 or embodiment 140, wherein the subject has failed two prior lines of standard of care therapy.

143. The anti-BAFFR antibody or binding fragment, method, combination or use according to embodiment 139 or embodiment 140, wherein the subject has failed three prior lines of standard of care therapy.

144. The anti-BAFFR antibody or binding fragment, method, combination or use according to embodiment 139 or embodiment 140, wherein the subject has failed four prior lines of standard of care therapy.

145. The anti-BAFFR antibody or binding fragment, method, combination or use according to embodiment 139 or embodiment 140, wherein the subject has failed five prior lines of standard of care therapy.

146. The anti-BAFFR antibody or binding fragment, method, combination or use according to any one of embodiments 139 to 145, wherein the at least one prior line of standard of care therapies comprise an anti-CD20 therapy.

147. The anti-BAFFR antibody or binding fragment, method, combination or use according to embodiment 146, wherein the anti-CD20 therapy is rituximab.

148. The anti-BAFFR antibody or binding fragment, method, combination or use according to any one of embodiments 139 to 147, wherein the subject is intolerant to or ineligible for one or more other approved therapies.

149. The anti-BAFFR antibody or binding fragment, method, combination or use according to embodiment 148, wherein the one or more other approved therapies comprise an autologous stem cell transplant (ASCT).

150. The anti-BAFFR antibody or binding fragment, method, combination or use according to any one of embodiments 139 to 149, wherein the subject is a non-responder to a CAR composition.

151. The anti-BAFFR antibody or binding fragment, method, combination or use according to embodiment 150, wherein the CAR composition is an anti-CD19 CAR composition.

152. The anti-BAFFR antibody or binding fragment, method, combination or use according to embodiment 150 or embodiment 151, wherein the CAR composition comprises CTL019, tisagenlecleucel, axicabtagene ciloleucel, brexucabtagene autoleucel or lisocabtagene maraleucel.

153. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is a Hodgkin's lymphoma.

154. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is multiple myeloma.

155. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is hairy cell leukemia.

156. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is primary effusion lymphoma.

157. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is B cell prolymphocytic leukemia.

158. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is plasmablastic lymphoma.

159. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is follicle center lymphoma.

160. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is precursor B-lymphoblastic leukemia.

161. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is high-grade B-cell lymphoma.

162. The anti-BAFFR antibody or a binding fragment thereof for use according to any one of embodiments 1 to 49, the method of any one of embodiments 50 to 100, the combination for use according to embodiment 111, or the use according to any one of embodiments 112 to 115, wherein the B cell malignancy is primary mediastinal large B-cell lymphoma.

163. The anti-BAFFR antibody or a binding fragment thereof for use according to any one of embodiments 1 to 49, the method of any one of embodiments 50 to 100, the combination for use according to embodiment 111, the use according to any one of embodiments 112 to 115, or the anti-BAFFR antibody or a binding fragment thereof for use, method, combination for use or use according to any one of embodiments 116 to 162, wherein the subject is not eligible for autologous stem cell transplant (ASCT) therapy.

164. The anti-BAFFR antibody or a binding fragment thereof for use according to any one of embodiments 1 to 49, the method of any one of embodiments 50 to 100, the combination for use according to embodiment 111, the use according to any one of embodiments 112 to 115, or the anti-BAFFR antibody or a binding fragment thereof for use, method, combination for use or use according to any one of embodiments 116 to 163, wherein the subject is not eligible for CAR-T therapy.

165. The anti-BAFFR antibody or a binding fragment thereof for use according to any one of embodiments 1 to 49, the method of any one of embodiments 50 to 100, the combination for use according to embodiment 111, the use according to any one of embodiments 112 to 115, or the anti-BAFFR antibody or a binding fragment thereof for use, method, combination for use or use according to any one of embodiments 116 to 164, wherein the subject does not meet any of the exclusion criteria set forth in Section 8.4.1.3.

166. The anti-BAFFR antibody or a binding fragment thereof for use according to any one of embodiments 1 to 49, the method of any one of embodiments 50 to 100, the combination for use according to embodiment 111, the use according to any one of embodiments 112 to 115, or the anti-BAFFR antibody or a binding fragment thereof for use, method, combination for use or use according to any one of embodiments 116 to 165, wherein the subject does not meet any of the exclusion criteria set forth in Section 8.4.1.4.

167. An anti-BAFFR antibody or a binding fragment thereof for use in the treatment of a subject with cytokine release syndrome (CRS) or at risk of CRS.

168. A method of reducing the severity of one or more symptoms of cytokine release syndrome (CRS) in a subject, comprising administering a therapeutically effective amount of an anti-BAFFR antibody or a binding fragment thereof to the subject.

169. Use of an anti-BAFFR antibody or a binding fragment thereof in the manufacture of a medicament for treating a subject with cytokine release syndrome (CRS) or at risk of CRS.

170. The anti-BAFFR antibody or a binding fragment thereof of embodiment 167, the method of embodiment 168, or the use of embodiment 169, wherein the anti-BAFFR antibody or a binding fragment thereof is an anti-BAFFR antibody or binding fragment thereof described in any one of embodiments 2 to 4.

### 10. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there are any inconsistencies between the teachings of one or more of the references incorporated herein and the present disclosure, the teachings of the present specification are intended.

What is claimed is:

1.-51. (canceled)

52. A method of treating a subject having a B cell malignancy, comprising administering therapeutically effective dose of an anti-BAFFR antibody or a binding fragment thereof to the subject.

53. The method according to claim 52, wherein the anti-BAFFR antibody or binding fragment thereof comprises CDR-H1, CDR-H2, and CDR-H3 having the amino acid sequences of SEQ ID NO:3, SEQ ID NO:4, and SEQ ID NO:5, and CDR-L1, CDR-L2, and CDR-L3 having the amino acid sequences of SEQ ID NO:6, SEQ ID NO:7, and SEQ ID NO:8, respectively.

54. The method according to claim 52, wherein the anti-BAFFR antibody or binding fragment thereof comprises a heavy chain variable region having the amino acid sequence of SEQ ID NO: 1 and a light chain variable region having the amino acid sequence of SEQ ID NO: 2.

55. The method according to claim 52, wherein the anti-BAFFR antibody or binding fragment thereof is ianalumab or a binding fragment thereof.

56. The method according to claim 52, wherein the anti-BAFFR antibody or binding fragment thereof is to be administered at a dose of 0.1 mg/kg to 20 mg/kg, 1 mg/kg to 10 mg/kg, 5 mg/kg to 15 mg/kg, or 10 mg/kg to 20 mg/kg.

57. The method according to claim 56, wherein the anti-BAFFR antibody or binding fragment thereof is to be administered at a dose of 1 mg/kg, 3 mg/kg, 6 mg/kg, 9 mg/kg, 12 mg/kg, or 20 mg/kg.

58. The method according to claim 52, wherein the anti-BAFFR antibody or binding fragment thereof is to be administered to a subject in need thereof once every two weeks (+/−3 days), once every week (+/−3 days), or once every 4 weeks (+/−3 days).

59. The method according to claim 52, wherein the anti-BAFFR antibody or binding fragment thereof is to be administered at a dose of 3 mg/kg, once every two weeks (+/−3 days).

60. The method according to claim 52, wherein the anti-BAFFR antibody or binding fragment thereof is to be administered at a dose of 9 mg/kg, once every four weeks (+/−3 days).

61. The method according to claim 52, wherein the anti-BAFFR antibody or binding fragment thereof is to be administered intravenously to a subject in need thereof.

62. The method according to claim 52, wherein the anti-BAFFR antibody or binding fragment thereof is to be administered as monotherapy for the B cell malignancy.

63. The method according to claim 52, wherein the anti-BAFFR antibody or binding fragment thereof is to be administered in combination with one or more additional agents.

64. The method according to claim 63, wherein the one or more additional agents comprise an immunomodulatory imide drug (IMiD).

65. The method according to claim 64, wherein the IMiD is lenalidomide or a pharmaceutically acceptable salt thereof, thalidomide or a pharmaceutically acceptable salt thereof, pomalidomide or a pharmaceutically acceptable salt thereof, or iberdomide or a pharmaceutically acceptable salt thereof.

66. The method according to claim 65, wherein the IMiD is lenalidomide or a pharmaceutically acceptable salt thereof.

67. The method according to claim 66, wherein the lenalidomide or a pharmaceutically acceptable salt thereof is to be administered at a dose of 2.5 mg to 25 mg.

68. The method according to claim 67, wherein the lenalidomide or a pharmaceutically acceptable salt thereof is to be administered at a dose of 2.5 mg, 5 mg, 15 mg, 20 mg, or 25 mg.

69. The method according to claim 66, wherein the lenalidomide or a pharmaceutically acceptable salt thereof is to be administered to a subject in need thereof once a day.

70. The method according to claim 52, wherein the B cell malignancy is a plasma cell dyscrasia, acute leukemia, B cell acute lymphocytic leukemia (B-ALL), non-Hodgkin's lymphoma (NHL), chronic lymphocytic leukemia (CLL)/small lymphocytic lymphoma (SLL), follicular lymphoma (FL), optionally wherein the FL is small cell FL or large cell FL, mantle cell lymphoma (MCL), diffuse large B-cell lymphoma (DLBCL), Burkitt lymphoma, lymphoplasmacytic lymphoma (Waldenstrom macroglobulinemia), MALT lymphoma (mucosa-associated lymphoid tissue lymphoma), marginal zone lymphoma (MZL), extranodal marginal zone lymphoma (EMZL), nodal marginal zone B-cell lymphoma (NZML), or splenic marginal zone B-cell lymphoma (SMZL).

71. The method according to claim 70, wherein the subject has failed at least one prior line of standard of care therapy.

72. The method according to claim 71, wherein the at least one prior line of standard of care therapies comprise an anti-CD20 therapy.
